(19) United States
(12) Patent Application Publication
(10) Pub. No.: US 2024/0366066 A1
(43) Pub. Date: Nov. 7, 2024

(54) DEVICES, SYSTEMS, AND METHODS FOR MOUNTING A MEDICAL DEVICE WITH RESPECT TO ANOTHER MEDICAL DEVICE

(71) Applicant: Boston Scientific Scimed, Inc., Maple Grove, MN (US)

(72) Inventors: John Mims, Austin, TX (US); Vladimir Mitelberg, Austin, TX (US); Jason Mehta, Austin, TX (US); Ryan Gilkey, Kyle, TX (US)

(73) Assignee: Boston Scientific Scimed, Inc., Maple Grove, MN (US)

(21) Appl. No.: 18/655,029

(22) Filed: May 3, 2024

Related U.S. Application Data
(60) Provisional application No. 63/541,150, filed on Sep. 28, 2023.
(60) Provisional application No. 63/464,007, filed on May 4, 2023.

(51) Int. Cl. A61B 1/00

(52) U.S. Cl. CPC A61B 1/00128

(57) ABSTRACT

An auxiliary system is provided for use with a medical device, the auxiliary system having a sheath configured to be mounted along the medical device. Systems are provided for rotationally aligning the distal end of the medical device relative to the distal mount of the auxiliary system. Additionally or alternatively, systems are provided for facilitating securing of the auxiliary system and other medical device relative to each other. For instance, systems are provided for securing the sheath along the length of the medical device, and subsequently releasing the secure coupling.

### CROSS-REFERENCE TO RELATED APPLICATIONS

This application claims the benefit of priority under 35 U.S.C. § 119 to U.S. Provisional Application No. 63/541,150, filed Sep. 28, 2023, and U.S. Provisional Application No. 63/464,007, filed May 4, 2023, the entire disclosures of which are hereby incorporated by reference herein for all purposes. Any and all priority claims identified in the Application Data Sheet, or any correction thereto, are hereby incorporated by reference under 37 C.F.R. § 1.57.

### FIELD

The present disclosure relates to devices, systems, and methods for mounting a medical device with respect to another medical device. The present disclosure further relates to additional or auxiliary devices and/or systems including devices and/or systems and associated methods for facilitating mounting of medical devices and/or systems with respect to another medical device. Devices, systems, and methods of the present disclosure may be particularly suitable for minimally invasive medical/surgical procedures.

### BACKGROUND

Various devices (e.g., instruments, tools, etc.) and systems for performing surgical operations or procedures are configured to be used with another device, such as a medical scope, and, more particularly, a pre-existing scope such as an endoscope (e.g., a commercially available endoscope, or endoscope otherwise not customized for the devices or systems to be used therewith). Such devices and systems may add bulk to the overall endoscopic system, which may present challenges during endoscopic procedures. Moreover, some such devices and systems must be used with a medical scope having two instrument channels, and may be limited to use with larger endoscopes with such features. However, smaller medical scopes are gaining favor for various reasons. For instance, smaller endoscopes, with their smaller profile, can be more easily advanced through a natural orifice, and are more easily advanced through tortuous body passages. Further devices and systems have been developed for use with smaller medical scopes having a single or no instrument channel. Such devices and systems may include an end cap adapted to receive the distal end of the scope and may be used with a scope independent of the number of instrument channels within the scope, including the smaller endoscopes that are available in many surgical settings and which can be more easily advanced through a natural orifice. However, improvements to the devices and manners of securing such systems to a medical scope or to another medical device would be welcome. Furthermore, it is generally important that the orientation of the medical scope be proper once the device or system is secured thereto so that imaging is consistent. In addition, different medical scopes have imaging lenses in different locations. Current systems do not provide a way to be registered consistently with respect to any of the available scopes in a suitable rotational orientation relative to the end cap without having the scope be active during attachment to the system. It is with respect to these and other considerations that the present improvements may be useful.

### SUMMARY OF THE DISCLOSURE

This Summary is provided to introduce, in simplified form, a selection of concepts described in further detail below in the Detailed Description. This Summary is not intended to necessarily identify key features or essential features of the claimed subject matter, nor is it intended as an aid in determining the scope of the claimed subject matter. One of skill in the art will understand that each of the various aspects and features of the present disclosure may advantageously be used separately in some instances, or in combination with other aspects and features of the disclosure in other instances, whether or not described in this Summary. No limitation as to the scope of the claimed subject matter is intended by either the inclusion or non-inclusion of elements, components, or the like in this Summary.

In accordance with various principles of the present disclosure, an auxiliary medical system is disclosed for use with another medical device having a proximal end and a distal end. The auxiliary system includes a flexible elongate member having a device-engaging section sized, shaped, configured, and/or dimensioned to engage the other medical device; a distal mount configured to engage the other medical device proximate to a distal end of the other medical device; and a proximal mount configured to engage the other medical device proximate to a proximal end of the other medical device.

In some aspects, the flexible elongate member is or includes a sheath having a length selected to extend at least partially along the length of the other medical device. In some aspects, the sheath has a device-engaging section with a concave shape configured to receive a convexly curved outer surface of the other medical device.

In some aspects, the distal mount is configured to mount on a distal end of a medical scope. In some aspects, the distal mount includes a distal stop against which the distal end of the medical scope is abutted when fitted within the distal mount, the distal stop including indicia relative to which a feature of the medical scope is rotationally alignable.

In some aspects, the flexible elongate member has one or more auxiliary working channels extending therethrough.

In some aspects, the auxiliary medical system further includes one or more attachment strips having a first end attachable to the flexible elongate member and wrappable around the flexible elongate member and the other medical device to secure the auxiliary medical system with respect to the other medical device. In some aspects, the auxiliary medical system further includes a tape system including the one or more attachment strips. In some aspects, the tape system further comprises a tape applicator. In some aspects, the distal mount includes hooks and at least one of the one or more attachment strips includes holes for securing over the hooks.

In some aspects, the proximal mount is configured to be coupled with a handle of the other medical device.

In accordance with various principles of the present disclosure, an auxiliary medical system is disclosed for use with another medical device having a proximal end and a distal end. The auxiliary system includes an auxiliary device configured to be mounted with respect to the other medical device; and at least one mount configured to engage the other medical device and an end of an attachment strip configured to be wrapped around the auxiliary device and the other medical device.

In some aspects, the at least one mount is an end cap configured to be mounted with respect to a distal end of the other medical device.

In some aspects, the at least one mount includes at least one hook configured to engage a hole in an end of an attachment strip configured to be wrapped around the auxiliary device and the other medical device. In some aspects, the auxiliary medical system further includes at least one attachment strip having a first end with a hole defined therethrough shaped to extend over the at least one hook of the at least one mount to secure the first end of the at least one attachment strip with respect to the at least one mount.

In accordance with various principles of the present disclosure, a method of engaging an auxiliary device with respect to another medical device is disclosed. The method includes engaging a section of the auxiliary device with the other medical device; and securing the auxiliary device with respect to the other medical device with two or more attachment strips by attaching a free end of the attachment strips to the auxiliary device and wrapping the attachment strips around the auxiliary device and the other medical device.

In some aspects, the auxiliary device is a sheath, and engaging a section of the sheath with the other medical device comprises engaging a concave section of the sheath with a convex surface of the other medical device.

In some aspects, the other medical device is a medical scope; the auxiliary device further includes a distal mount configured to be mounted on a distal end of the medical scope; and the method further includes comprising orienting features on the distal mount of the auxiliary device with respect to features of the medical scope.

In some aspects, securing the auxiliary device with respect to the other medical device further includes tightening at least one of the attachment strips while wrapping the at least one attachment strip after securing the free end of the at least one attachment strip with respect to the auxiliary device. In some aspects, the auxiliary device further includes a distal mount configured to be mounted with respect to a distal end of the other device, the method further including hooking a hole formed in the free end of the at least one of the attachment strips over a hook extending from the distal mount.

These and other features and advantages of the present disclosure, will be readily apparent from the following detailed description, the scope of the claimed invention being set out in the appended claims. While the following disclosure is presented in terms of aspects or embodiments, it should be appreciated that individual aspects can be claimed separately or in combination with aspects and features of that embodiment or any other embodiment.

### BRIEF DESCRIPTION OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. For example, devices may be enlarged so that detail is discernable, but is intended to be scaled down in relation to, e.g., fit within a working channel of a delivery catheter or endoscope. For purposes of clarity and simplicity, not every element is labeled in every figure, nor is every element of each embodiment shown where illustration is not necessary to allow those of ordinary skill in the art to understand the disclosure.

The detailed description will be better understood in conjunction with the accompanying drawings, wherein like reference characters represent like elements, as follows:

FIG. 1 is an elevational view of a device and system formed in accordance with various principles of the present disclosure.

FIG. 2 is a cross-sectional view along line II-II of FIG. 1.

FIG. 3 is a perspective distal end view of a cap assembly formed in accordance with various principles of the present disclosure mounted and properly oriented with respect to another medical device.

FIG. 4 is a view similar to that of FIG. 3, showing the cap assembly and other medical device being positioned with respect to each other.

FIG. 5 is a schematic illustration of a taping system for securing a device and/or system formed in accordance with various principles of the present disclosure relative to another device.

FIG. 6 is a schematic illustration of a tape applicator which may be used in conjunction with the taping system illustrated in FIG. 5.

FIG. 7 is a perspective view of an endoscopic suturing system according an embodiment of the invention.

FIG. 8 is a proximal perspective view of a suturing device of the endoscopic suturing system of FIG. 7.

FIG. 9 is a side elevation view of a suturing device, of indefinite length, of the endoscopic suturing system of FIG. 7.

FIG. 10 is a perspective distal end view of a cap assembly attached at the distal end of an endoscope of the endoscope suturing system.

FIG. 11 is a perspective distal end view of a cap assembly of the suturing device relative to an endoscope for the endoscopic suturing system.

FIG. 12 is a cross section view across line XII-XII in FIG. 7.

FIG. 13 is a schematic distal end view of the cap assembly of the suturing device relative to an endoscope for the endoscopic suturing system.

FIG. 14 is a view similar to FIG. 13 with the endoscope rotated into alignment.

FIG. 15 is a view similar to that of FIG. 11 with the endoscope shown properly oriented and advanced with respect to the distal end of a cap assembly.

FIG. 16 is a schematic illustration of a tape applicator.

FIG. 17 is a schematic illustration of a taping system for securing a sheath of the suturing device relative to the endoscope.

FIG. 18 and FIG. 19 illustrate features of the tape of the tape system.

FIG. 20 and FIG. 21 are schematic illustrations of a retroflex system for the endoscopic system, as applied at the proximal handle of the system.

FIG. 22 is a schematic illustration of a detail of one embodiment of a lock for the retroflex system.

FIG. 23 is a schematic illustration of the endoscopic system subject to distal retroflex as applied by the retroflex system.

FIG. 24 is a schematic illustration of a detail of operation of the lock of FIG. 22.

FIGS. 25-29 illustrate use of the tape applicator to apply tape at the cap assembly to secure the cap assembly relative to the distal end of the endoscope.

FIG. 30 illustrates use of the tape along the sheath to secure the sheath to the endoscope.

FIGS. 31-35 illustrate use of the endoscopic system to endoscopically suture tissue.

FIG. 36 and FIG. 37 illustrate disassembly of the tape from about the cap assembly.

FIG. 38 illustrates disassembly of the sheath from the endoscope.

FIG. 39 and FIG. 40 illustrate examples of alignment of a cap assembly as in FIG. 13 and FIG. 14.

### DETAILED DESCRIPTION

The following detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more principles of this disclosure. Each example of an embodiment is provided by way of explanation and is not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. In fact, it will be apparent to those skilled in the art that various modifications and variations can be made in the present disclosure without departing from the scope or spirit of the present subject matter. For instance, features illustrated or described as part of one embodiment can be used with another embodiment to yield a still further embodiment. Thus, it is intended that the present subject matter covers such modifications and variations as come within the scope of the appended claims and their equivalents.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

As used herein, “proximal” refers to the direction or location closest to the user (medical professional or clinician or technician or operator or physician, etc., such terms being used interchangeably herein without intent to limit, and including automated controller systems or otherwise), etc., such as when using a device (e.g., introducing the device into a patient, or during implantation, positioning, or delivery), and/or closest to a delivery device, and “distal” refers to the direction or location furthest from the user, such as when using the device (e.g., introducing the device into a patient, or during implantation, positioning, or delivery), and/or closest to a delivery device. “Longitudinal” means extending along the longer or larger dimension of an element. A “longitudinal axis” extends along the longitudinal extent of an element, though is not necessarily straight and does not necessarily maintain a fixed configuration if the element flexes or bends, and “axial” generally refers to along the longitudinal axis. However, it will be appreciated that reference to axial or longitudinal movement with respect to the above-described systems or elements thereof need not be strictly limited to axial and/or longitudinal movements along a longitudinal axis or central axis of the referenced elements. “Central” means at least generally bisecting a center point and/or generally equidistant from a periphery or boundary, and a “central axis” means, with respect to an opening, a line that at least generally bisects a center point of the opening, extending longitudinally along the length of the opening when the opening comprises, for example, a tubular element, a channel, a cavity, or a bore. As used herein, a “lumen” or “channel” or “bore” or “passage” is not limited to a circular cross-section. As used herein, a “free end” of an element is a terminal end at which such element does not extend beyond. It will be appreciated that terms such as at or on or adjacent or along an end may be used interchangeably herein without intent to limit unless otherwise stated, and are intended to indicate a general relative spatial relation rather than a precisely limited location. As understood herein, “corresponding” is intended to convey a relationship between components, parts, elements, etc., configured to interact with or to have another intended relationship with one another. Finally, reference to “at” a location or site is intended to include at and/or about the vicinity of (e.g., along, adjacent, proximate, etc.) such location or site.

It will be appreciated that various principles of the present disclosure may be applied to a variety of medical procedures, particularly surgical procedures. Such surgical procedures may be performed manually (e.g., with various instruments being controlled with or by the hands of the medical professional(s) performing the procedure) and/or robotically (e.g., with an automated device, such as a robotic device, controlling the various instruments being used to perform the procedure, with the automated device, in turn, being controlled by the medical professional). Moreover, although various embodiments may be described herein as relating to endoscopic procedures, it will be appreciated that the principles of the present disclosure need not be so limited.

In accordance with various principles of the present disclosure, devices and/or systems are mounted with respect to a medical device. More particularly, the devices and/or systems are mounted with respect to a medical device such as a medical scope, including, without limitation, an endoscope. In some aspects, the devices and/or systems are configured to modify, augment, improve, or otherwise, the medical device with respect to which the devices and/or systems are mounted. It will be appreciated that terms such as modify, augment, improve, etc., and other grammatical forms thereof, may be used interchangeably herein without intent to limit. In accordance with various principles of the present disclosure, the devices and/or systems modify an existing device, such as a medical scope. As referenced herein, “existing” is intended to cover commercially available, pre-existing, off-the-shelf, or otherwise available, and not customized, modified, etc., for the devices and/or systems described herein. As such, the devices and/or systems of the present disclosure may be considered to be universally applicable to a type or category of medical device with respect to which the devices and/or systems are to be mounted (e.g., medical scopes), rather than designed for a particular design, configuration, brand, etc. In some aspects, devices and/or systems of the present disclosure augment the functionality of another medical device or system. For instance, in accordance with various principles of the present disclosure, devices and/or systems of the present disclosure provide additional working channels for a medical scope, such as by providing exterior working channels over the scope. Principles of the present disclosure may be particularly beneficial for use with medical devices and/or systems for performing minimally invasive surgical procedures, such as transluminal, transcatheter, endoscopic, etc., procedures which do not require open surgery (cutting open the patient), but, instead, access a target site within the patient via a natural orifice (or, in some instances, a small incision not considered to constitute an open-surgery cut).

In accordance with various further principles of the present disclosure, devices and/or systems are disclosed herein which facilitate mounting of devices and/or systems with respect to another medical device. In some aspects, devices and/or systems disclosed herein as facilitating mounting of other devices and/or systems also are configured to facilitate dismounting or removal of such other devices and/or systems.

Various embodiments of devices and/or systems mounted with respect to a medical device, and associated methods, as well as devices and/or systems facilitating mounting of devices and/or systems with respect to a medical device, and associated methods, will now be described with reference to examples illustrated in the accompanying drawings. Reference in this specification to “one embodiment,” “an embodiment,” “some embodiments”, “other embodiments”, etc. indicates that one or more particular features, structures, concepts, and/or characteristics in accordance with principles of the present disclosure may be included in connection with the embodiment. However, such references do not necessarily mean that all embodiments include the particular features, structures, concepts, and/or characteristics, or that an embodiment includes all features, structures, concepts, and/or characteristics. Some embodiments may include one or more such features, structures, concepts, and/or characteristics, in various combinations thereof. It should be understood that one or more of the features, structures, concepts, and/or characteristics described with reference to one embodiment can be combined with one or more of the features, structures, concepts, and/or characteristics of any of the other embodiments provided herein. That is, any of the features, structures, concepts, and/or characteristics described herein can be mixed and matched to create hybrid embodiments, and such hybrid embodiment are within the scope of the present disclosure. Moreover, references to “one embodiment,” “an embodiment,” “some embodiments”, “other embodiments”, etc. in various places in the specification are not necessarily all referring to the same embodiment, nor are separate or alternative embodiments necessarily mutually exclusive of other embodiments. It should further be understood that various features, structures, concepts, and/or characteristics of disclosed embodiments are independent of and separate from one another, and may be used or present individually or in various combinations with one another to create alternative embodiments which are considered part of the present disclosure. Therefore, the present disclosure is not limited to only the embodiments specifically described herein, as it would be too cumbersome to describe all of the numerous possible combinations and subcombinations of features, structures, concepts, and/or characteristics, and the examples of embodiments disclosed herein are not intended as limiting the broader aspects of the present disclosure. It should be appreciated that various dimensions provided herein are examples and one of ordinary skill in the art can readily determine the standard deviations and appropriate ranges of acceptable variations therefrom which are covered by the present disclosure and any claims associated therewith. The following description is of illustrative examples of embodiments only, and is not intended as limiting the broader aspects of the present disclosure.

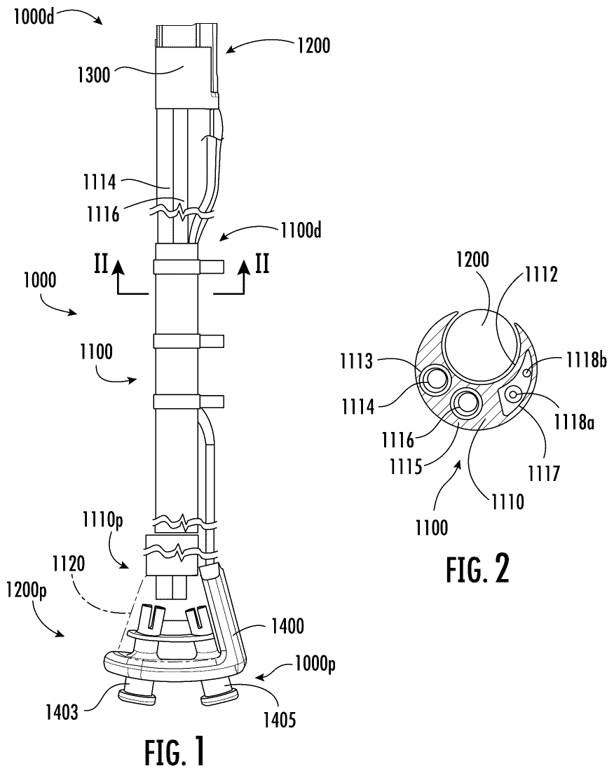

Turning now to the drawings, an example of an embodiment of a system 1000 for mounting an auxiliary device 1100 with respect to a medical device 1200 in accordance with various principles of the present disclosure is illustrated in FIG. 1. The illustrated example of an embodiment of an auxiliary device 1100 includes a sheath 1110 adapted to accommodate a medical device 1200 with respect to which the system 1000 and auxiliary device 1100 are mounted. In the illustrated example of an embodiment, the medical device 1200 is an endoscope. However, principles of the present disclosure are applicable with respect to other medical devices, the present disclosure not being limited to use with endoscopes. In some embodiments, the auxiliary device 1100 is configured to be mounted with respect to the medical device 1200, such as by having a device-engaging section thereof. The device-engaging section of the auxiliary device 1100 may be any section of the auxiliary device 1100 sized, shaped, configured, and/or dimensioned to engage, receive, be mounted with respect to, or otherwise be coupled with respect to the medical device 1200, such as with respect to an outer surface (e.g., a side surface) of a medical device 1200 It will be appreciated that terms such as engage, receive, mount, couple, connect, attach, associate with, operatively associate with, etc., including other grammatical forms thereof, may be used interchangeably herein without intent to limit unless otherwise indicated. The system 1000 may include additional structures, features, devices, etc., to facilitate maintaining the auxiliary device 1100 with respect to the medical device 1200 (e.g., holding the auxiliary device 1100 with respect to the medical device 1200), as described in further detail below.

As noted above, in the example of an embodiment of an auxiliary device 1100 illustrated in FIG. 1, the auxiliary device 1100 includes a sheath 1110. In some aspects, the device-receiving mounting section of the auxiliary device 1100 is defined and extends along a portion of the sheath 1110. For instance, as illustrated in the cross-sectional view of FIG. 2, along line II-II of FIG. 1, at least a section of the sheath 1110 includes a concave recess 1112 sized, shaped, configured, and/or dimensioned to facilitate mounting of the sheath 1110 with respect to the medical device 1200. For instance, the concave recess 1112 defined along a device-receiving mounting section of the sheath 1110 may be sized, shaped, configured, and/or dimensioned so that the sheath 1110 may extend in close approximation to the exterior of the medical device 1200. For instance, the curvature of the concave recess 1112 may be selected to fit against and/or to receive the exterior of any of a variety of commercially-available endoscopes. In some aspects, the concave recess 1112 may extend more than 180° about a circumference of, and thereby partially surround the exterior of, the medical device 1200. In some aspects, the concave recess 1112 extends along a portion of the length of the sheath 1110 or along substantially the entire length of the sheath 1110, such as to facilitate navigation of the medical device 1200 with the system 1000 mounted with respect thereto.

In accordance with various principles of the present disclosure, the auxiliary device 1100 may further be configured to facilitate extension of one or more additional devices, tools, instruments, components, etc., along the system 1000. In some embodiments, the auxiliary device 1100 defines one or more auxiliary working channels to supplement the one or more working channels of the medical device 1200. In the cross-sectional view of an example of an embodiment of an auxiliary device 1100 illustrated in FIG. 2, the sheath 1110 of the auxiliary device 1100 defines one or more auxiliary working channels 1113, 1115, such as by having one or more lumens defined longitudinally therethrough and defining working channels through which instruments, tools, devices, components, etc. (such terms being used interchangeably herein without intent to limit) may be inserted, advanced, retracted, etc. The one or more auxiliary working channels 1113, 1115 provide additional working channels to the overall system to supplement one or more working channels of the medical device 1200. In some aspects, an elongate tubular member 1114, 1116 (e.g., catheter, tube, sheath, etc.) may be provided in one or both of the auxiliary working channels 1113, 1115, respectively, to define working channels through which further devices are inserted, advanced, retracted, etc. The elongate tubular members 1114, 1116 may be separately formed, discrete elements, or may be formed by separate lumens of a common elongate tubular member, the present disclosure not being limited in this regard. In embodiments in which elongate tubular member 1114, 1116 are provided within the auxiliary working channels 1113, 1115, when the sheath 1110 is mounted with respect to the medical device 1200, the elongate tubular members 1114, 1116 are mounted with respect to the medical device 1200 as well, and extend longitudinally along the medical device 1200 to facilitate advancement or retraction of devices therethrough. In some embodiments, the sheath 1110 extends along substantially all (e.g., at least 80 percent, or at least 90 percent) of the length of the elongate tubular members 1114, 1116, with an end (e.g., terminal, free end) of one or both of the elongate tubular members 1114, 1116 extending beyond a respective distal or proximal end 1110d, 1110p of the sheath 1110. However, the present disclosure need not be so limited, and the sheath 1110 may extend along the entire length of the elongate tubular members 1114, 1116, a partial length thereof, or may be provided in sections along selected portions of the elongate tubular members 1114, 1116.

In some embodiments, a portion of one or both of the elongate tubular members 1114, 1116 are fixed with respect to the auxiliary working channels 1113, 1115 in which the elongate tubular member 1114, 1116 extends. For instance, one or both of the elongate tubular members 1114, 1116 may be affixed along one or more points along the auxiliary working channels 1113, 1115 defined through the sheath 1110. In some aspects, the system 1000 includes a distal mount 1300 at a distal end 1000d thereof configured to be mounted with respect to the medical device 1200 (e.g., with respect to a distal end 1200d of the medical device 1200), and the distal end of one or both of the elongate tubular members 1114, 1116 is fixed with respect to such distal mount 1300. In some aspects, the distal end of at least one of the elongate tubular members 1114, 1116 extends through a throughbore defined through the distal mount 1300, and is affixed thereto. In the example of an embodiment illustrated in FIG. 1, the elongate tubular members 1114, 1116 extend beyond a distal end 1100d of the sheath 1110 and along the medical device 1200 before extending into respective throughbores in the distal mount 1300. In some aspects, the system 1000 includes a proximal mount 1400 at a proximal end 1000p thereof configured to be mounted with respect to the medical device 1200 (e.g., with respect to a proximal end 1200p of the medical device 1200). In some aspects, the proximal end of one or both of the elongate tubular members 1114, 1116 are fixed with respect to an adaptor 1120 at (e.g., mounted on) the proximal end 1100p of the auxiliary device 1100. In some aspects, the adaptor 1120 includes access ports 1403, 1405 via which the lumens through the respective elongate tubular members 1114, 1116 may be accessed.

In accordance with various principles of the present disclosure, the above-mentioned distal mount 1300 is a structure or device sized, shaped, configured, and/or dimensioned to be mounted with respect to a medical device 1200. In some aspects, the distal mount 1300 is configured to be mounted with respect to a distal portion of the medical device 1200. In some aspects, the distal mount 1300 is an end cap or other structure with features, components, devices, etc., providing further functionality, such as including structures and/or devices for performing a procedure with respect to a body, such as body tissue. In some aspects, the distal mount 1300 has at least a section thereof configured to engage, receive, be mounted with respect to, etc., a portion of the medical device 1200. In some aspects, such section allows removable engagement of the distal mount 1300 and the portion of the medical device 1200. In some embodiments, the distal mount 1300 is shaped to extend a sufficient amount around the circumference/perimeter of the medical device 1200 to remain mounted thereon once engaged with the medical device 1200. For example, the distal mount 1300 may include a peripheral engagement structure sized, shaped, configured, and/or dimensioned to be positioned approximately 180°, and preferably slightly greater than 180°, around a medical device 1200. In some embodiments, the engagement structure includes a concave recess 1312 sized, shaped, configured, and/or dimensioned to facilitate mounting of the distal mount 1300 with respect to the medical device 1200, such as with respect to the distal end 1200d of the medical device 1200. In some embodiments, the distal mount 1300 may be considered a clip which engages the medical device 1200 with a friction or interference fit. In some aspects, the distal mount 1300 is formed of a resilient material which allows the distal mount 1300 to be released from the medical device 1200, such as upon completion of the procedure (e.g., to allow sterilization and reuse of the medical device 1200). In some aspects, system 1000 includes separate elements to further secure the distal mount 1300 with respect to the medical device 1200, and optionally to facilitate removal of the distal mount 1300 after use of the system 1000, as described in further detail below.

In accordance with various principles of the present disclosure, it may be desirable to orient the system 1000, such as the sheath 1110 and/or the distal mount 1300 thereof, with respect to one or more features or structures of the medical device 1200. For instance, if the system 1000 is configured to guide an instrument to a distal end 1200d of the medical device 1200, it may be desirable for such device to be positioned at a particular location with respect to a working channel of the medical device 1200. Additionally or alternatively, if the medical device 1200 is a medical scope with optical systems (e.g., visualization systems such as a camera, optical fiber, etc.), it may be desirable for any devices, structures, etc., delivered by or otherwise associated with respect to the system 1000 to be positioned so as not to interfere with the functioning of the optical system of the medical device 1200.

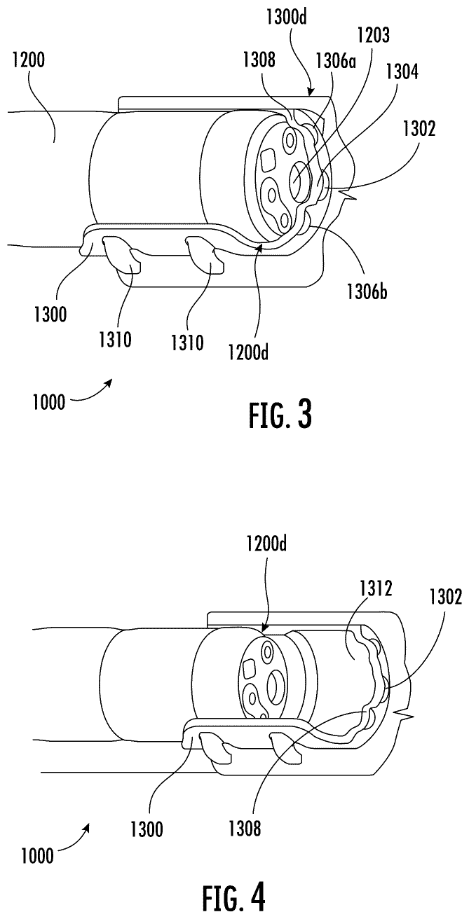

In accordance with various principles of the present disclosure, the distal mount 1300 of the system 1000 of the present disclosure may include one or more alignment features structured, formed, shaped, positioned, or otherwise, to facilitate alignment of the distal mount 1300 with respect to a medical device 1200. In some aspects, the alignment feature includes a structure with respect to which a user may be guided in rotationally orienting the system 1000 with respect to a medical device 1200. In some aspects, the structure is formed along a periphery/circumference of the distal mount 1300. In some aspects, the structure is provided along the distal end 1300d of the distal mount 1300. In the example of an embodiment of a distal mount 1300 of the example of an embodiment of a system 1000 illustrated in FIG. 3, the alignment feature includes indicia 1302 and/or a recess 1304 along a perimeter of the distal mount 1300, optionally along a distal end 1300d of the distal mount 1300.

In some aspects, indicia 1302 on a distal mount 1300 formed in accordance with various principles of the present disclosure may provide visual aide in rotationally aligning the distal mount 1300 with respect to the medical device 1200. In some aspects, the indicia 1302 is a contrasting color relative to a color of the portion of the distal mount 1300 on which the indicia 1302 is provided.

In some aspects, a recess 1304 on a distal mount 1300 formed in accordance with various principles of the present disclosure not only facilitates orientation of the distal mount 1300 with respect to the medical device 1200, but is also sized, shaped, configured, and/or dimensioned to be positioned with respect to a feature of the medical device 1200 to allow clearance for functionality of one or more features of the medical device 1200. For instance, the recess 1304 may be positioned and sized, shaped, configured, and/or dimensioned to permit pass through access to or from the working channel 1203 of any of a variety of a medical devices 1200 in the form of a commercially available endoscope of any of a variety of manufacturers when the distal mount 1300 is properly rotationally aligned with respect to the medical device 1200. In some aspects, the recess 1304 may be considered a clearance window. In some embodiments, additional (optionally smaller) recesses 1306a, 1306b (which may be considered additional clearance windows), may be provided along the perimeter/circumference of the distal mount 1300, and optionally along the distal end 1300d of the distal mount 1300. The illustrated examples of embodiments of additional recesses 1306a, 1306b are positioned on either side of the recess 1304, such as to provide clearance for other features or structures of the medical device 1200. For instance, the additional recesses 1306a, 1306b may be positioned and sized, shaped, configured, and/or dimensioned to prevent obstruction of a visualization system (e.g., the optical lens) of a medical device 1200 in the form of a medical scope, and/or other working features of various medical devices such as medical scope (e.g., endoscopes).

In some aspects, the indicia 1302 may be used to facilitate alignment of the one or more recesses 1304, 1306a, 1306b with respect to the medical device 1200. For example, the indicia 1302 may be aligned with a feature of the medical device 1200, such as a working channel 1203 or visualization system (e.g., (e.g., camera, optical lens, etc.) of a medical device 1200 in the form of a medical scope. With such rotational alignment of the system 1000 of the present disclosure with respect to the medical device 1200, the various features or systems of the medical device 1200 are assured unobstructed use. For instance, alignment of the indicia 1302 provided on the distal mount 1300 of a system 1000 formed in accordance with various principles of the present disclosure with a medical device 1200 in the form of a medical scope may assure unobstructed view and appropriate orientation of a visualization system (e.g., camera, optical lens, etc.) relative to further tools, devices, instruments, etc., advanced and utilized at and/or distal to the distal end 1200d of the medical device 1200, regardless of the specific details, structure, configuration, etc., of the medical device 1200 selected (e.g., among variety of medical devices of a variety of manufacturers) for use with the system 1000 of the present disclosure. In some aspects, the relative orientation, access, field of view, etc., of the medical device 1200 can be assured/ascertained without requiring powering up the medical device 1200, such as powering up a medical scope and previewing the field of view of the scope (e.g., the field of view from the lens on the display associated with the scope) in advance of performing the procedure with the scope and the system 1000 of the present disclosure. This significantly reduces preparation time prior to the procedure.

In some embodiments, to further facilitate placement of a system 1000 formed in accordance with various principles of the present disclosure with respect to a medical device 1200, the distal mount 1300 includes a stop 1308. The stop 1308 may be positioned, and sized, shaped, configured, and/or dimensioned to facilitate longitudinal orientation of the system 1000 with respect to the medical device 1200. For instance, the stop 1308 may be positioned, and sized, shaped, configured, and/or dimensioned to facilitate longitudinal orientation of the distal mount 1300 with respect to the distal end 1200d of the medical device 1200. In the example of an embodiment illustrated in FIG. 3 and FIG. 4, the stop 1308 is formed at or along the distal end 1300d of the distal mount 1300. In some aspects, the stop 1308 is positioned and sized, shaped, configured, and/or dimensioned to inhibit or prevent the distal end 1200d of the medical device 1200 from extending beyond the longitudinal position of the stop 1308. In the example of an embodiment illustrated in FIG. 4, the stop 1308 extends radially-inwardly into a concave recess 1312 formed in the distal mount 1300 to inhibit or prevent the distal end 1200d of the medical device 1200 from extending beyond the longitudinal position of the stop 1308. As may be appreciated with reference to FIG. 4, the distal end 1200d of the medical device 1200 may be extended into the recess 1312 of the distal mount 1300 and advanced longitudinally towards the stop 1308 until seated against the stop 1308. Such relative longitudinal positioning of the distal mount 1300 with respect to the medical device 1200 may also facilitate use of various devices, instruments, components, of the system 1000 of the present disclosure and/or advanced to a treatment site with the use of the medical device 1200 with respect to which the system 1000 is associated. It will be appreciated that rotational and longitudinal alignment may be performed in any order (rotational alignment performed before or after longitudinal alignment) or together (in conjunction with each other, aligning rotationally and longitudinally in harmony).

In accordance with various principles of the present disclosure, the above-mentioned proximal mount 1400, configured to facilitate association of a system 1000 formed in accordance with various principles of the present disclosure with a medical device 1200, is a structure or device sized, shaped, configured, and/or dimensioned to be mounted with respect to a medical device 1200. In some aspects, the proximal mount 1400 is configured to be mounted with respect to a proximal portion of the medical device 1200. The proximal mount 1400 may be configured or otherwise include a section sized, shaped, configured, and/or dimensioned to mount the system 1000 with respect to a handle of the medical device 1200. In some aspects, the proximal mount 1400 has at least a section thereof configured to engage, receive, be mounted with respect to, etc., a portion of the medical device 1200, and to remain mounted with respect thereto once engaged. In some aspects, such section allows removable engagement of the proximal mount 1400 and the portion of the medical device 1200, such as to allow removal upon completion of the procedure (e.g., to allow sterilization and reuse of the medical device 1200). In some aspects, the proximal mount 1400 may be a bracket with one section thereof coupled to the auxiliary device 1100 and another section thereof coupled to the medical device 1200. However, the present disclosure need not be limited in this regard.

As may be appreciated, a system 1000 formed in accordance with various principles of the present disclosure may increase the functionality of a system utilizing a medical device 1200 without compromising the functionality of the medical device 1200. In particular, the auxiliary working channels 1113, 1115, and the optional one or more elongate tubular member 1114, 1116, augment or supplement the working channels of a pre-existing medical device 1200 with respect to which the system 1000 is mounted. For instance, the medical device 1200 with the system 1000 associated therewith is not limited by the size (e.g., inner diameter) and/or number of working channels of the medical device 1200 since additional working channels are provided by the system 1000. Each of the auxiliary working channels 1113, 1115 provided by a system 1000 formed in accordance with various principles of the present disclosure may facilitate advancement and use of a device, tool, instrument, etc., alongside the medical device 1200 with respect to which the system 1000 is associated, leaving the working channel of such medical device 1200 to be used for other purposes or functions. In some aspects, a sheath 1110 formed in accordance with various principles of the present disclosure may be provided with additional structures facilitating advancement and/or retraction of further components, devices, tools, instruments, etc., with respect to a medical device 1200. For instance, in the example of an embodiment illustrated in FIG. 2, the sheath 1110 may include one or more additional lumens or auxiliary working channels 1117 sized, shaped, configured, and/or dimensioned to allow passage of additional components, tools, devices, instruments, therethrough. Depending on the overall cross-sectional shape, material, and other properties of the sheath 1110, the additional auxiliary working channels 1117 may allow passage of more than one additional component, tool, device, instrument, etc., therethrough, or may include a separate additional auxiliary working channel for each component, tool, device, instrument, etc. For instance, in the example of an embodiment illustrated in FIG. 2, the illustrated additional auxiliary working channel 1117 allows passage of both a first elongate element 1118a and a second elongate element 1118b. The elongate elements 1118a, 1118b may be longitudinally translatable through the auxiliary working channel 1117 and formed of materials which do not interfere with each other. Optionally, separate lumens can be provided for the elongate elements 1118a, 1118b.

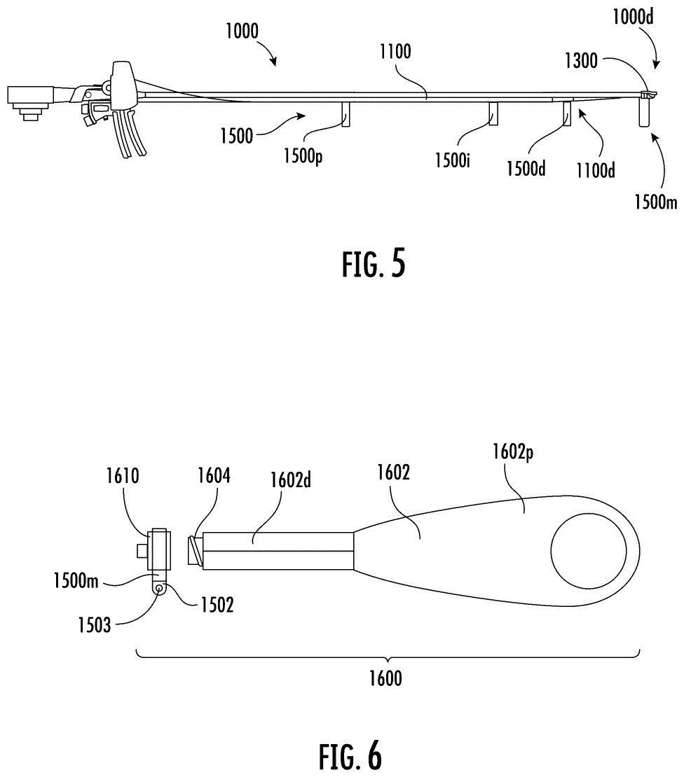

As noted above, a system 1000 formed in accordance with various principles of the present disclosure may include additional structures, features, devices, etc., to facilitate maintaining the auxiliary device 1100 with respect to the medical device 1200 (e.g., holding the auxiliary device 1100 with respect to the medical device 1200). In an example of an embodiment of a system 1000 illustrated in FIG. 5, a system 1000 formed in accordance with various principles of the present disclosure includes a plurality of attachment strips 1500 sized, shaped, configured, and/or dimensioned to facilitate maintaining one or more components of the system 1000 with respect to a medical device 1200. For instance, in the example of an embodiment of a system 1000 illustrated in FIG. 5, the attachment strips 1500 are provided along a length of the system 1000 to secure and/or to facilitate maintaining of at least a portion of the sheath 1100 relative to a medical device 1200. The sheath 1100 may be provided with a plurality of attachment strips 1500 pre-disposed along the length of the sheath 1100. In the example of an embodiment illustrated in FIG. 5, three attachment strips 1500d, 1500p, 1500i are provided: a distal attachment strip 1500d located adjacent a distal end 1100d of the sheath 1100; a proximal attachment strip 1500p positioned along a proximal end 1100d of the sheath 1100, or approximately midway or proximal to a position midway along the length of the sheath 1110; and one or more intermediate attachment strips 1500i positioned between the distal attachment strip 1500a and the proximal attachment strip 1500p. In some embodiments, as illustrated, the intermediate attachment strip 1500i is positioned closer to the distal attachment strip 1500d than to the proximal attachment strip 1500p. However, other positions are within the scope of the present disclosure. The positioning of two or three attachment strips 1500 spaced apart from one another in accordance with various principles of the present disclosure has been shown to provide good stabilization of the sheath 1100 relative to the medical device 1200 with respect to which the system 1000 is associated, particularly during use of the system 1000 and/or medical device 1200. That said, additional attachment strips 1500 can be provided along the length of the sheath 1100 and used in the manner described.

In accordance with various principles of the present disclosure, the attachment strips 1500 may be in the form of any of a variety of strips or other elongated elements capable of securing and/or maintaining a portion of the system 1000 (e.g., the sheath 1110 thereof) with respect to a medical device 1200. For instance, the attachment strips 1500 may be formed of a band of material (e.g., a flat band), tape, cord, or other preferably elongated clement. In some aspects, the attachment strips 1500 have a length selected to allow wrapping of the attachment strips 1500 at least once around the peripheral extent (e.g., circumference) of a portion of the system 1000 and the medical device 1200. In some aspects, the attachment strips 1500 may be wrapped slightly more than once (greater than) 360° around the peripheral extent of a portion of the system 1000 and the medical device 1200. In some embodiments, the attachment strips 1500 are formed of tape having an adhered portion pre-applied to the sheath 1100; an intermediate portion that is non-sticky to prevent adhesion to the medical device 1200 as the attachment strips 1500 is wrapped around the medical device 1200; and an end portion extending from the intermediate portion to a free, terminal end of the attachment strip 1500 that carries an adhesive on at least a portion thereof and is adapted to adhere to the portion of the tape which has been wrapped around the system 1000 and the medical device 1200.

In some embodiments, one or more of the attachment strips include a pull-off protective strip over one or more portions of the attachment strip 1500, such as to cover adhesive thereon. The pull-off protective strip may include a free end configured to facilitate removal of the pull-off protective strip from the portion of the attachment strip 1500 (e.g., an L-shaped tab). In some embodiments, the tab includes a high visibility color, such as to contrast with the sheath 1100, and/or indicia, such as indicating the direction in which the protective strip should be pulled to be released from the attachment strip 1500 to expose the adhesive.

Additionally or alternatively, one or more of the attachment strips 1500 are configured to facilitate removal from the system 1000 after use of the system 1000. In some embodiments, one or more of the attachment strips 1500 may be cut with readily available blades/scissors, and/or may have at least one perforation line or other type of line-of-weakness facilitating breakaway disassembly of the system 1000 from the medical device 1200.

In some embodiments, an attachment strip 1500m is provided to secure and/or to maintain a distal mount 1300 of the system 1000 with respect to a medical device 1200, such as in the example of an embodiment illustrated in FIG. 5. The attachment strip 1500m may have the same configuration as the attachment strips 1500 provided along the sheath 1110 or a different configuration, the present disclosure not being limited in this regard.

In the example of an embodiment of a distal mount 1300 illustrated in FIG. 3, the distal mount 1300 includes one or more structures 1310 facilitating securing and/or coupling of one or more attachment strips 1500m with respect to the distal mount 1300. For instance, in the example of an embodiment illustrated in FIG. 3, the distal mount 1300 includes one or more longitudinally spaced apart hooks 1310. In some embodiments, the hooks 1310 are identified with contrasting color relative to surrounding areas of the distal mount 1300, and/or are numbered with indicia, e.g., “1” and “2” (such as according to the intended order of use). In some embodiments, the attachment strips 1500m have apertures through a portion (e.g., proximal portion) thereof which are secured with respect to the distal mount 1300 such as by being extended over the hooks 1310 on the distal mount 1300. Once mounted with respect to the distal mount 1300, the attachment strips 1500m may be pulled and wound about the distal mount 1300 and the medical device 1200 to assure a tight and firm connection therebetween.

In some embodiments, a system 1000 formed in accordance with various principles of the present disclosure also includes a tape applicator 1600 configured to facilitate application of attachment strips 1500m with respect to the distal mount 1300. The example of an embodiment of a tape applicator 1600 illustrated in FIG. 6 includes a handle 1602 with a proximal end 1602p configured to be grasped by a user, and a distal end 1602d with respect to which a spool 1610 carrying an attachment strip 1500m may be mounted with respect to the handle 1602. The distal end 1602d of the handle 1602 may include a narrowed extension with a spool mount 1604 formed on a distal end thereof. In some aspects, the attachment strip 1500m has a non-adhesive free terminal end 1502 through which an aperture or loop 1503 is formed. The aperture or loop 1503 is sized, shaped, configured, and/or dimensioned for mounting with respect to one of the hooks 1310 on the distal mount 1300 (such as described above with reference to FIG. 3) to facilitate a secure connection between the attachment strip 1500m and the distal mount 1300. In some embodiments, the loop 1503 is reinforced to withstand pulling force applied to the attachment strip 1500m when wrapping the attachment strip 1500m around the distal mount 1300 and the medical device 1200 to assure a secure connection therebetween. In some embodiments, the loop 1503 is provided in a color to facilitate identification thereof (e.g., locating the loop 1503 in the environment of an operating room).

Various principles of the present disclosure such as those described above may be implemented in a variety of manners and/or applied to a variety of medical devices, examples of which will now be described. It will be appreciated that various methods described with respect to the described examples of embodiments may be applied to other embodiments, such as described more broadly/generically above.

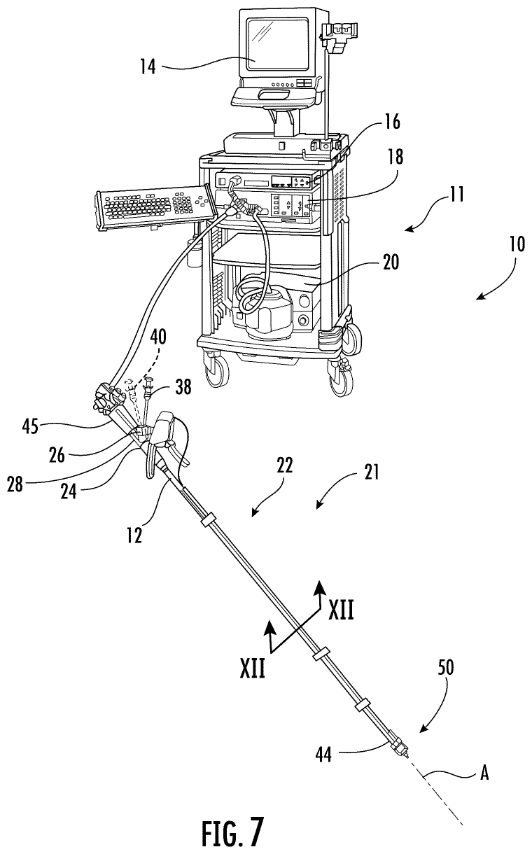

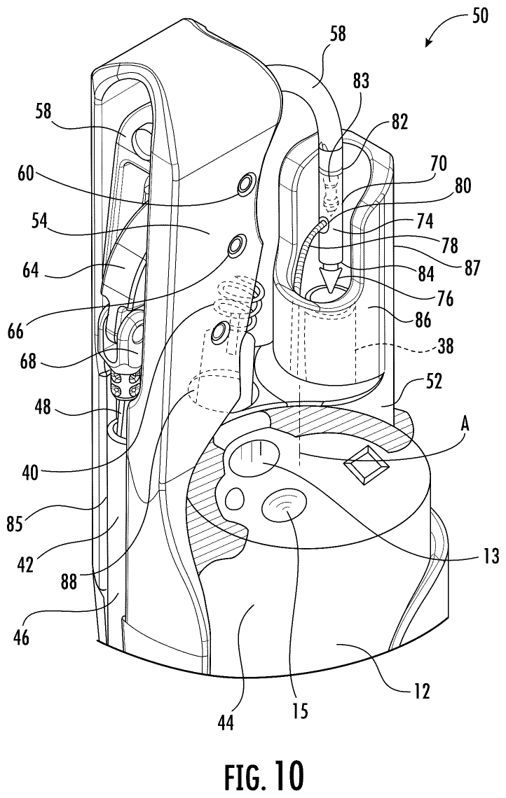

Referring to FIGS. 7-10, an endoscopic treatment system 10 includes an endoscope system 11 and an endoscopic suturing system 21. The endoscope system 11 includes an endoscope 12, a video display unit 14, an image processing device 16, a light source 18, and a suction device 20. In accordance with an embodiment, the endoscope 12 has a small profile, generally 5-10 mm in diameter. However, the size of the endoscope is not critical, and elements described herein can be adapted for endoscopes of other sizes. In the example of an embodiment shown, the endoscope 12 has a single instrument channel 13 and an optical lens 15 (FIG. 10). However, the endoscope 12 may have more than one instrument channel or no instrument channel all, as operation of the system does not necessarily require use of the instrument channel through the endoscope. The endoscope 12 includes a distal end 44 and a proximal end 45 and a longitudinal axis A extending therebetween.

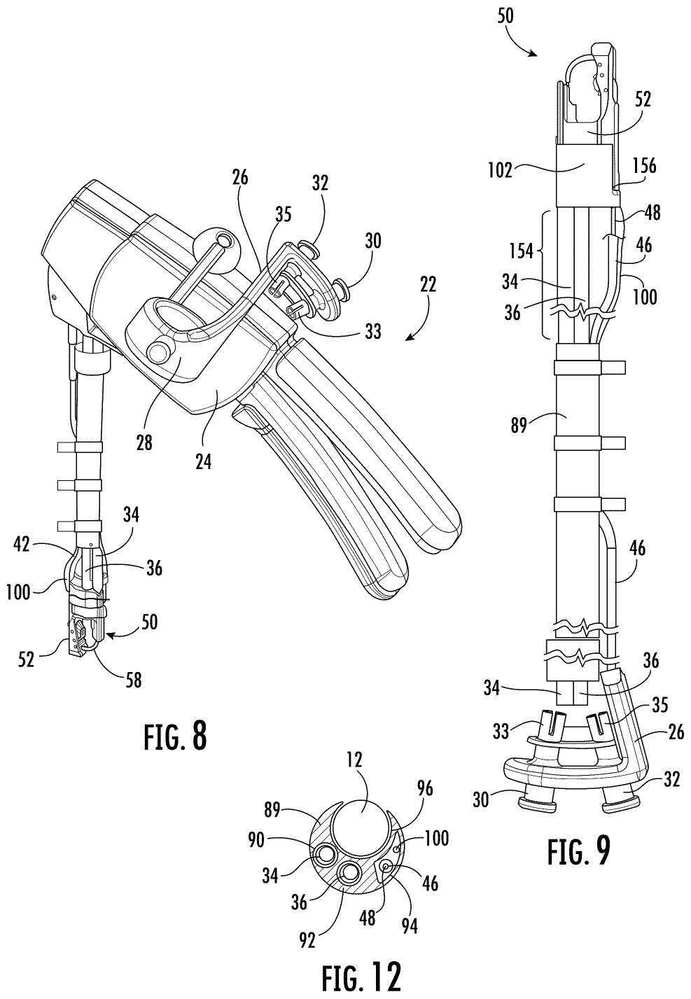

The suturing system 21 includes a suturing device 22 (FIG. 8), a needle assembly 70 (FIG. 10) movable through tissue by the suturing device 22, and first and second devices 38, 40 used in association with the suturing device 22 (FIG. 10).

Referring to FIGS. 7-9, the suturing device 22 has a proximal operable handle 24 provided with a mounting bracket 26 and a collar 28 at which the handle is removably coupled to endoscope 12. The bracket 26 includes first and second instrument ports 30, 32 at which instruments can be received into first and second catheters 34, 36, respectively. First and second tubular connectors 33, 35 are aligned with the ports 30, 32 that couple the ports 30, 32 to the first and second catheters 34, 36.

Referring to FIG. 8, FIG. 9, and FIG. 12, a transmission assembly 42 includes a transmission sheath 46 and a transmission cable 48 displaceable within the transmission sheath 46, both coupled relative to the handle 24. The transmission sheath 46 is coupled relative to a first portion of the handle (i.e., a stationary member), and the transmission cable 48 is coupled to a second portion of the handle (i.e., a movable lever), such that when the handle 24 is operated the cable 48 is displaced within the transmission sheath 46.

The first and second catheters 34, 36 and the transmission assembly 42 extend from the proximal handle 24, along the outside of the endoscope 12, to a distal cap assembly 50. The distal cap assembly 50 is adapted to be mounted at the distal end 44 of the endoscope 12, and the handle 24 remotely operates the cap assembly 50 via the transmission assembly 42.

Referring to FIG. 10, the cap assembly 50 includes a mount 52, U-shaped support bracket 54 extending distally from the mount, and a needle arm 58 rotatably mounted on the bracket 54 with a first pin 60. A bell crank 64 is rotatably mounted at a second pin 66 on the support bracket 54 and engages the needle arm 58 at intermeshing gears (not shown). The distal end of the transmission cable 48 of the transmission assembly 42 is attached to the bell crank 64 at a clevis 68. When the transmission assembly 42 is operated by the handle 24, it results in rotation of the bell crank 64 and consequent rotation of the needle arm 58 between the open and closed positions.

The needle assembly 70 is coupled to a needle mount 83 at an end of the needle arm 58. The needle assembly 70 includes a tubular needle body 74, a needle tip 76, and suture 78 coupled to the needle body. The needle body 74 includes a side opening 80 through which the suture 78 extends, a first end 82 at which the needle assembly is coupled to the needle mount 83, and a second end 84 to which the tip 76 is coupled. The tip 76 defines a tissue-piercing taper. The suture 78 may be formed of any materials commonly available for surgical suture such as nylon, polyolefins, PLA, PGA, stainless steel, nitinol, and others. One suitable needle assembly is described in more detail in U.S. Pat. No. 9,198,562, which patent is hereby incorporated herein by reference in its entirety for all purposes.

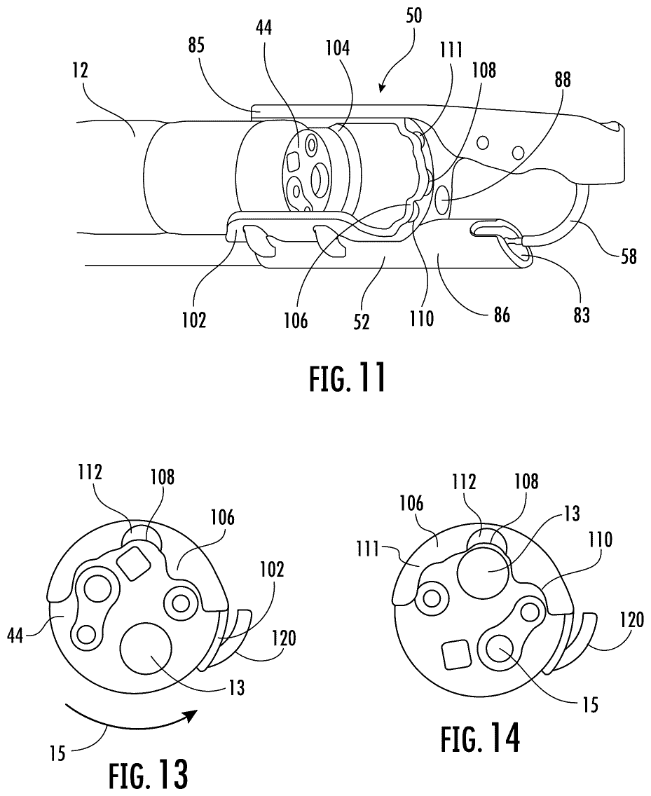

Referring to FIG. 10 and FIG. 11, the mount 52 of the cap assembly 50 includes a side recess 85 into which the transmission assembly 42 is received, and a first throughbore 86 and a second throughbore 88. The first throughbore 86 is positioned in alignment with both the needle mount 83 of the needle arm 58 and needle assembly 70 when the needle arm 58 is in the closed position. A tissue guide 87 extends distally on the mount 52 from over the first throughbore 86 and provides a surface on which to stabilize tissue as it is pierced by the needle assembly 70. The second throughbore 88 is positioned between the first throughbore 86 and the support bracket 54. More particularly, the axial center of the second throughbore 88 is positioned between the first throughbore 86 and the pin 60 (or axis) on which the end effector and/or assembly 58 rotates. The first and second throughbores 86, 88 may be parallel to each other and the longitudinal axis A of the endoscope, or the second throughbore 88 may be obliquely angled relative to the first throughbore 86 so as to direct the second device 40 at a particular orientation into the needle path, as described further below. The mount 52 is structured such that when the cap assembly 50 is coupled to the endoscope 12, as described below, the first and second throughbores 86, 88 are positioned radially outside the profile of the endoscope 12.

Referring to FIG. 9 and FIG. 12, the distal end of the first catheter 34 is fixed in the first throughbore 86, and its proximal end is coupled to a first connector 33 on the handle bracket 26. The distal end of the second catheter 36 is fixed in the second throughbore 88, and its proximal end is coupled the second connector 35 on the handle bracket 26. The first and second catheters 34, 36 may be discrete catheters or may be separate lumens of a common catheter. In a preferred embodiment, the catheters 34, 36 are discrete catheters covered in a preferably crescent moon shaped common sheath 89 along substantially all (e.g., at least 80 percent, and more preferably at least 90 percent) of their length. Alternatively, the common sheath 89 may extend along the entire length of the catheters 34, 36, a partial length thereof, or may be provided in sections along selected portions of the catheters 34, 36. In a preferred embodiment, the common sheath 89 is flexible, defines a lumen 90 for catheter 34, a lumen 92 for catheter 36, and a lumen 94 for both transmission assembly 42 and a retroflex pull string 100, the function of which is described below. Optionally, separate lumens can be provided for the transmission assembly 42 and the pull string 100. A concave recess 96 is provided along the length of the sheath 89 so that the sheath is preferably adapted to extend in close approximation to the exterior of an appropriately sized endoscope 12. In an embodiment, the sheath 89 is adapted to accommodate the endoscope such that the sheath preferably extends more than 180° about a circumference of, and thereby partially surrounds the exterior, of the appropriately sized endoscope 12.

Referring to FIG. 9 and FIG. 10, the first catheter 34 is adapted to receive a first device 38 that has a distal end effector that can receive and grasp the needle assembly 70. The second catheter 36 is adapted to receive a second device 40 that has a distal end effector that can engage tissue, and draw the tissue back into the path of the needle so that the tissue can be pierced by the needle assembly 70 as the needle assembly 70 is moved from the open to the closed position.

Referring to FIG. 11, the cap assembly 50 is secured to the distal end 44 of the endoscope 12 with a peripheral engagement structure that is adapted to be positioned about approximately 180°, and preferably slightly greater than 180°, around the circumference of the distal end of the endoscope 12. In one embodiment, the structure is a resilient clip 102 integrated at the proximal end of the mount 52. The clip 102 includes an opening 104 to allow the distal end 44 of the endoscope 12 access therethrough. The clip 102 may be formed from ABS plastic, other suitable plastics, elastic materials, as well as polymer-coated metals.

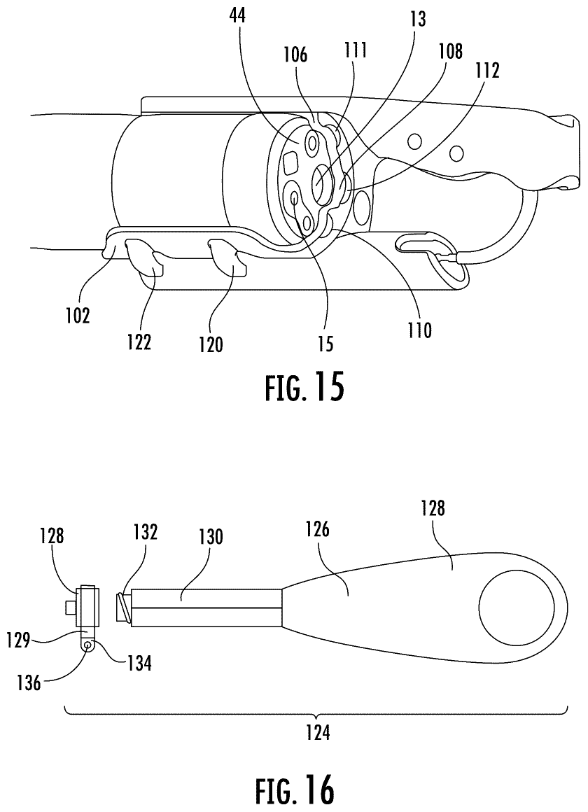

Turning to FIG. 11, FIG. 13, FIG. 14, and FIG. 15, a stop 106 is formed at the distal end of the clip 102. The distal end 44 of the endoscope 12 is adapted to pushed into the recess 104 of the clip 102 and seat against the stop 106. The stop 106 preferably includes a largest first clearance window, formed as a first recess 108 in the circumference of the stop 106 to permit pass through access to instrument channel 13 on various endoscopes of multiple manufacturers when the endoscope is properly rotationally aligned in the clip 102, and smaller second and third clearance windows, formed as second and third recesses 110, 111 approximately 60° on either side of the first recess 108, to prevent obstruction of the optical lens 15 and other working features of various endoscopes. The first recess 108 is located radially inward of the instrument channel lumen 88. In order to align the features of the endoscope relative to such first, second and third recesses 108, 110, 111, the stop 106 includes an indicia 112 relative to which the instrument channel 13 or other designated feature of an endoscope is rotationally aligned. For example, if an endoscope does not include an instrument channel 13 (as such is not required with the system described herein), the optical lens could be designated to be aligned with the indicia 112. In a preferred embodiment, the indicia 112 is a contrasting color relative to a color of a majority of the cap assembly which is provided about the first recess 108. With such rotational alignment of the designated endoscope feature relative to the indicia 112 (in the direction of arrow 115), the optical lens 15 of the endoscope is assured unobstructed view and appropriate orientation relative to the suturing arm 58, instrument channel lumen 88, and needle exchange lumen 86, regardless of the major manufacturer endoscope utilized with the system, without necessitating activating the endoscope. That is, the relative orientation, access, and field of view can be ascertain without requiring powering up the endoscope and previewing the field of view from the lens 13 on the display 14 in advance of the procedure. This significantly reduces preparation time prior to the procedure. The location of the first and second recesses 108, 110 and the indicia 112 relative to the suturing arm 58, instrument channel lumen 88, and needle exchange lumen 86, as well as areas which will be covered and blocked from view at the distal end of the endoscope, is based on analyses of scans of multiple distal ends of endoscopes.

Referring to FIG. 15, the resilient clip 102 further includes a plurality of longitudinally displaced (proximal and distal) integrated hooks 120, 122. In an embodiment, exactly two hooks are provided to the clip. The hooks are preferably identified with contrasting color relative to surrounding areas of the end cap and to a majority of the cap assembly. The hooks 120, 122 may also be numbered with indicia, e.g. “1” and “2”, according to the intended order of use, as further described below.

Turning to FIG. 16, the system also includes a tape applicator 124 for applying tape at each of the hooks 120, 122. The tape applicator 124 includes a handle 126 and a spool 128 of tape 129 on a distal end of the handle. The illustrated example of an embodiment of a handle 126 is preferably monolithic, including a wider proximal end 126p for stable gripping in a user's hand, and a narrower extension 130 for unobtrusively reaching about the distal end 44 of the endoscope 12 and cap assembly 50 during tape application. The extension 130 includes a spool mount 132. In a preferred embodiment the spool mount 132 is in the form of an external thread. The spool 128 is rotatably fixed on the spool mount 132 of the handle 126 during use. The spool 128 includes an internal thread (not shown) that allows the spool to be rotated onto the spool mount 132 in a rotational direction opposite the intended application of the tape, such that when tension is applied to the tape the spool is pulled in a direction that rotationally secures the spool relative to the mount. Other securing interfaces between the mount and spool can be used. The tape preferably has a non-adhesive free end 134 provided with an integrated loop 136 that is reinforced and sized for attachment to one of the hooks 120, 122. The reinforced integrated loop 136 is preferably provided in a color to facilitate locating it in the environment of an operating room. By way of example, the loop 136 is preferably opaque; and the loop preferably matches color with the hooks on the cap assembly. Both may be opaque white. In an embodiment, the loop 136 has a diameter of approximately 2.5 mm. The diameter of the spool 128 is preferably less than the diameter of the endoscope 12 to provide mechanical advantage when wrapping the tape 129 to secure the clip 102 and endoscope 12 relative to each other. In one embodiment, the spool has a diameter of 10 mm and a tape width of 5 mm. In an embodiment, except for the free end 134, the tape 129 has a consistent adhesion along its length and is primarily adapted to stick to the back of itself under tension, as well as both the endoscope 12 and the clip 102. Alternatively, the tape 129 can have a non-stick first portion for first wrapping about the endoscope, and a second adhesive portion for wrapping over and adhering to the back of the first portion and the back of the second portion.

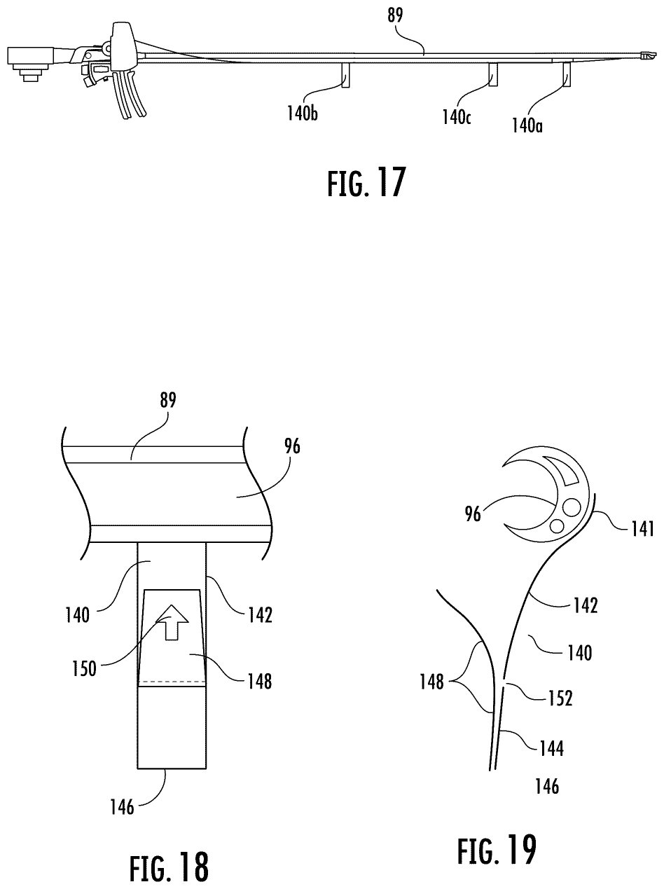

Turning now to FIG. 17, FIG. 18, and FIG. 19, a tape system is also provided for securing a portion of the sheath relative to the endoscope. The sheath 89 is provided with a plurality of strips of tape 140 pre-disposed along the length of the sheath 89. In a preferred embodiment, three strips of tape are used; a first strip 140a located at a distal end of the sheath, a second strip 140b located at a substantially central location along the length of the sheath, and a third strip 140c located between the first and second strips 140a, 140b and preferably closer to the first strip 140a. This limited use of tape and particular spacing has been shown to provide good stabilization of the sheath relative to the endoscope during use. That said, additional strips of tape can be pre-provided to the sheath and used in the manner described. In accordance with one aspect of the system, the strips of tape 140 have an adhered first portion 141 pre-applied to the sheath 89, a second portion 142 close to the sheath recess 96 that is non-sticky to prevent adhesion to the endoscope as the tape is wrapped around the endoscope, and a third portion 144 extending from the second portion 142 and to a free end 146 of the tape strip that is adhesive and adapted to adhere to the back of the tape when it is wrapped around the endoscope 12 and sheath 89. In accordance with another aspect, the tape strips 140 each include a pull off protective strip 148 that covers the second portion 144. The pull off protective strip 148 extends to cover the third portion 144 of the tape and is bent into an L-shaped extending tab between the free end 146 and the first portion 141. The tab 148 is preferably a high visibility color, contrasting with the sheath, e.g., white, and also preferably includes indicia, e.g., a laser-cut arrow 150, indicating the direction in which the tab 148 of the protective strip should be pulled to release the protective strip from the third portion to expose the adhesive (FIG. 18). In accordance with one aspect of the system, the strips 140 have at least a perforation 152 that permits breakaway disassembly after use of the system (FIG. 19). The perforation 152 is preferably provided at or near the junction of the second and third portions 142, 144 of the tape.

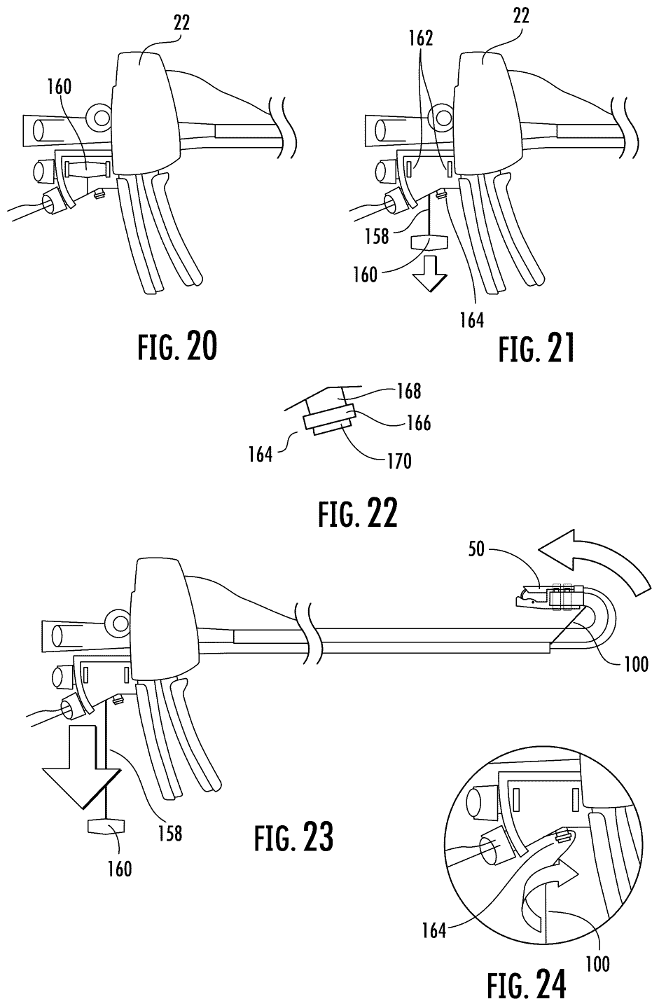

Turning to FIG. 9, FIG. 20, and FIG. 21, in accordance with another aspect of the system, the cap assembly 50 is adapted to retroflex relative to the sheath 89 via actuation from the proximal handle 24. The pull string 100, referenced above, extends from the proximal handle 24, through lumen 94 in the sheath 89, and exits the distal end of the sheath. A pull string 100 herein refers to a textile, polymer, or metal monofilament, multifilament, or cable stranded clement. Between the distal end of the sheath and the end cap there is gap 154 (FIG. 9) exposing a length of the catheters 34, 36, the transmission assembly 42, and the pull string 100 outside the sheath 89 and to the cap assembly 50. The length of the gap 154 generally represents the difference in length between the endoscope 12, which has a longer first length, and the sheath 89, which has a shorter second length. In a preferred embodiment the exposed length of the gap 154 is less than 16 cm; more preferably the length of the gap is between 6 and 12 cm; even more preferably the length of the gap is between 7.5 and 10 cm; and most preferably the exposed length of the gap is 8.5 cm to provide optimal retroflexion, as described below. On the opposite side of the gap 154, the transmission assembly 42 is secured in the side recess 85 of the cap assembly 50 and the catheters 34, 36 are secured within the first and second throughbores 86, 88 (e.g., as illustrated in FIG. 10). Further, a distal end 156 of the pull string 100 is secured to the cap assembly 50. In one assembly, the distal end 156 is bonded into a hole (not shown) at the proximal end of the cap assembly 50. The proximal end 158 of the pull string 100 exits from the sheath and through an opening in the proximal handle 24 and is attached to a pull handle 160. A pull handle mount 162 is preferably formed on the proximal handle 24 and can secure the pull handle 160 via interference fit when not in use. A pull string lock 164 is provided on the proximal handle 24 to secure the pull string in a tensioned state. An example of an embodiment of a pull string lock 164 includes a resilient rubber washer 166 secured on a seat 168 and over a post 170 (FIG. 22). In use, when it is desirable to retroflex the cap assembly 50, the pull handle 160 can be released from the handle mount 162 and pulled to apply tension to the pull string 100. As tension is applied to the pull string 100, the pull string 100 is retracted across the gap 154 and through the lumen 94. As the pull string 100 is pulled across the gap 154, the cap assembly 150 is retroflexed to shorten the distance between the end of the sheath 89 and the location at which the distal end 156 of the string is attached to the cap assembly 50, as shown in FIG. 23. When a desired degree of retroflex is obtained, the tension on the pull string 100 may be temporarily maintained (and the retroflex configuration fixed) by wrapping the proximal end 158 of the pull string 100 around the post 170 between the washer 166 and the scat 168 (FIG. 24). Compression of the pull string 100 between the washer 166 and the seat 168 retains the pull string in position. The pull handle 160 can be placed back on the handle mount 162 to keep it out of the way (i.e., back to the position shown in FIG. 20) while other features of the proximal handle 24 and the endoscope 12 are used. Retroflex of the system may be used in combination with actuating retroflex of the endoscope. Then, when it is intended to release the retroflex condition at the distal end, the proximal end 158 of the pull string 100 is unwound from the pull string lock 164, and tension on the pull string is released. In such manner, retroflex of the system operates as an assist to the system attached to the endoscope and ensures that retroflex of the endoscope is carried out full in the manner intended. It is also recognized that this type of retroflex system, with or without assist from the endoscope, can be used in different endoscopic therapeutic and interventional systems, and is not limited to systems for suturing.

In light of the above, the suturing device may be prepared for use in conjunction with an endoscope as follows. The distal end 44 of the endoscope 12 is pushed into the resilient clip 104 until seated at or near the distal stop 106 (FIG. 11). Then, the endoscope and cap assembly 50 are coaxially rotated relative to each other until the instrument channel 13 of the endoscope is rotationally aligned with the indicia 112 at the distal stop 106 to ensure appropriate alignment between the two (FIG. 13 and FIG. 14). Then, if necessary, the endoscope 12 is pushed all the way into contact with the distal stop 106 (FIG. 15).

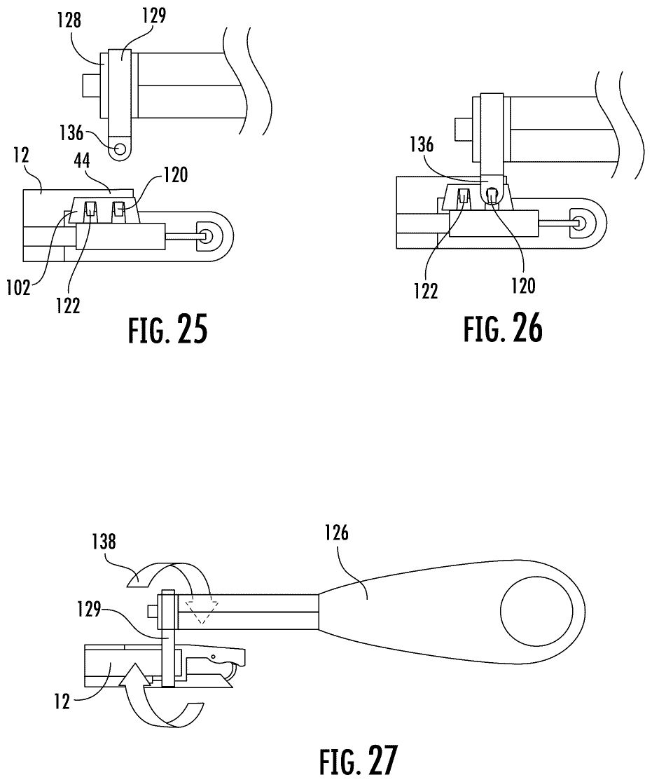

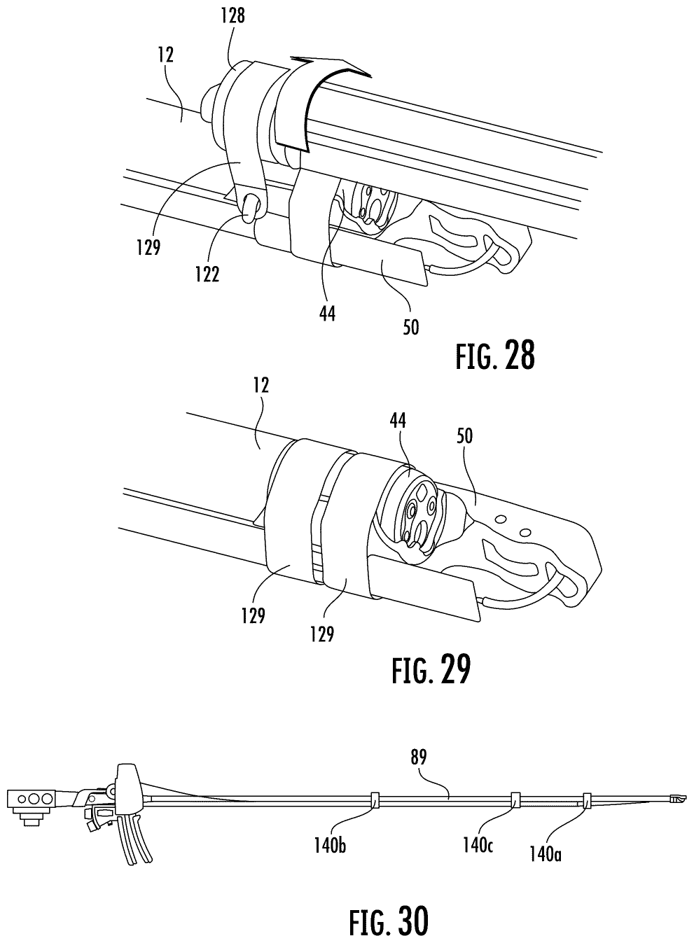

Then, referring to FIG. 25, FIG. 26, and FIG. 27, the loop 136 at the free end 134 of a first spool 128 of a tape applicator 124 is engaged over the distal first hook 120 and the handle 126 coupled to the spool 128 is maneuvered to wrap the tape 129 over a distalmost portion of the distal end 44 of the endoscope 12, over the clip 102, and back and around over the back of the tape 129 in a circular motion (arrow 138) one or more times to secure the endoscope relative to the clip. While wrapping, tension is applied to the tape. The relative diameters of the tape spool 128 and the endoscope 12 facilitate application of significant tension on the tape during wrapping of the endoscope with the tape. After the tape is applied at the first hook 120, the depleted spool 128 is removed from the spool mount 132, and a fresh spool 128 of tape is attached at the spool mount of the handle 126. Referring to FIG. 28 and FIG. 29, the tape 129 from the fresh spool 128 is similarly applied starting from the second hook 122 and over an adjacent area of the endoscope 12 until the distal end 44 of the endoscope 12 and cap assembly 50 are secured together.

Then, turning to FIG. 12, FIG. 18, FIG. 19, and FIG. 30, the endoscope is fully pressed into the recess 96 along the length of the sheath 89. Starting from the distal end of the system, and at strip of tape 140a, the tab 148 of the protective strip is pulled off to expose the adhesive portion 144, the tape 140 is pulled taut, the first portion 142 of the tape is wrapped around the endoscope 12, and the second adhesive portion 144 of the tape is adhered to the sheath on the opposite side of the recess 96 and then extended over the tape again to secure the endoscope thereat. The process is repeated along the length of the sheath for remaining strips 140c, then 140b, to properly secure the sheath relative to the endoscope. This longitudinally secures the sheath 89 to the exterior of the endoscope 12 for in vivo operation.

The collar 28 is properly positioned at the proximal handle 45 of the endoscope 12 (FIG. 7). The first device, a needle capture instrument 38 loaded with a needle assembly 70, is advanced through the first port 32, into the first lumen 34 and to the cap assembly 50. Suitable needle capture devices 38 are described in detail in U.S. Pat. No. 8,679,136, which patent is hereby incorporated herein by reference in its entirety for all purposes. The needle assembly 70 is loaded onto the needle arm 58, with the suture 78 extending parallel to the needle capture instrument 38 within the first lumen 34.

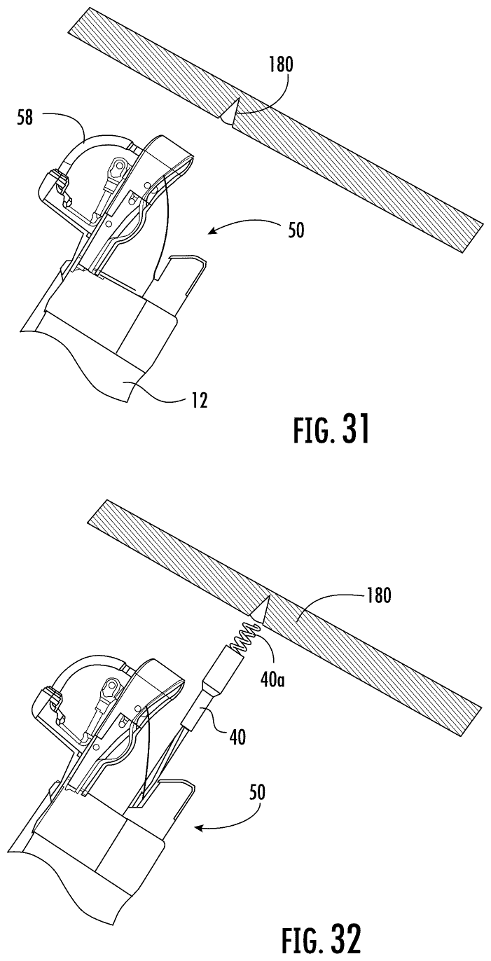

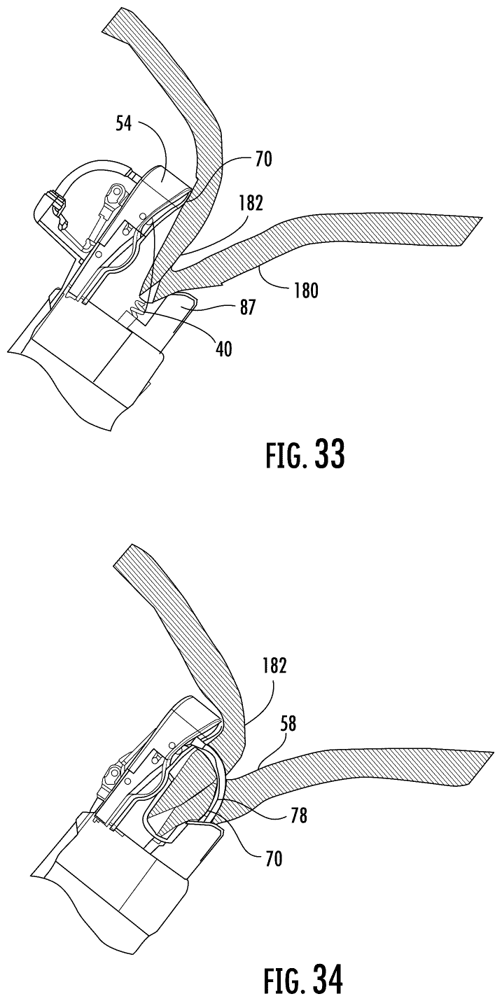

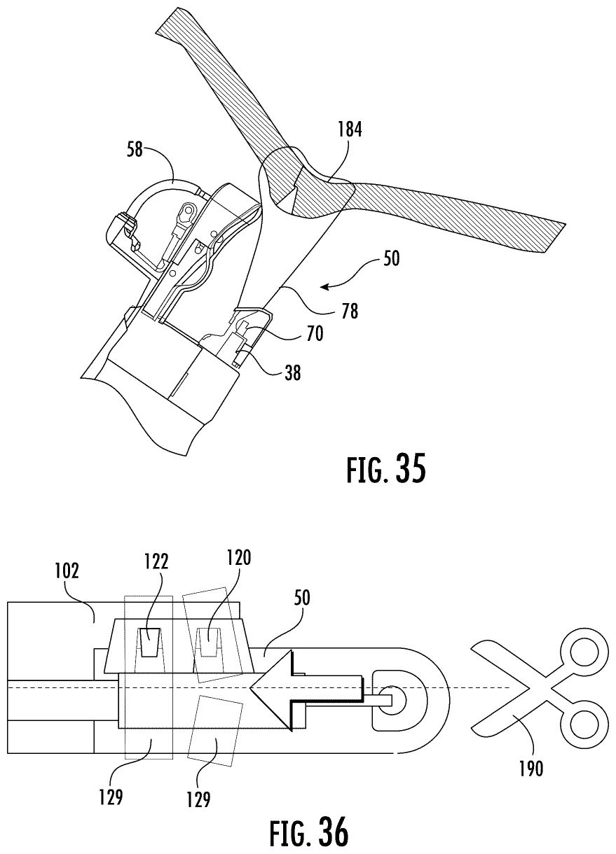

With reference to FIG. 7 and FIG. 31, the distal end of the endoscope 12 and cap assembly 50 of the suturing device 22 are advanced into a natural orifice of a patient, optionally through a guide tube (not shown), and approached to target tissue 180. If at any point it is necessary to reorient the endoscope 12 via retroflexion, the cap assembly 50 can be retroflexed as well, as described above, to aid and/or further direct the distal end of the endoscope and the cap assembly to the intended target tissue. Once adjacent the target tissue 180, the handle 24 of the suturing device 22 is operated to move the needle arm 58 into the open position, as shown in FIG. 30. Turning to FIG. 32, the end effector of the second device, e.g., a tissue retractor 40 having a helical coil 40a at its distal end, is advanced through the second port 32, into the second catheter 36 (FIG. 9) and out the second throughbore 88 (FIG. 10), and beyond the cap assembly 50. Suitable tissue retractor instruments are described in detail in U.S. Pat. No. 11,083,364, which patent is hereby incorporated herein by reference in its entirety for all purposes. Other tissue retractors, including forceps, may also be used. The helical coil 40a is operated to engage target tissue 180. The tissue retractor 40 is withdrawn to draw the tissue 180 against the tissue guard 87 and into a tissue fold 182 located within the path of the needle assembly 70; i.e., between the bracket 54 and needle guide 87, as shown in FIG. 33. The orientation of the second throughbore 88, either parallel or obliquely angled relatively to the first throughbore 86, is adapted to guide the tissue retractor to engage and retract tissue into the needle path. The handle 24 is then operated to move the needle arm 58 into the closed position, thereby piercing the tissue fold 182 and passing the needle assembly 70 with suture 78 through the tissue fold during the movement (FIG. 34). When the needle arm 58 is in the closed position, the needle is received within the distal end of the needle capture device 38 (FIG. 10). The needle capture device 38 is operated to securely engage the needle 70. The handle 24 is then operated to move the needle arm 58 toward the open position, thereby disengaging the needle arm 58 from the needle assembly 70, which remains in the needle capture device 38 (FIG. 35). The tissue retractor 40 is also released from the tissue and withdrawn back through the second catheter 36. The endoscope 12 is then moved to displace the cap assembly 50 relative to the sutured tissue 184. The needle 70 and suture 78 may be secured onto the tissue, such as by knotting or cinching, or the needle may be repositioned on the needle arm and additional suture loops may be formed within adjacent or other areas of tissue. Once the suturing is complete, the needle arm 58 is returned to a closed position, and the endoscope 12 and suturing device 22 are removed from the patient.

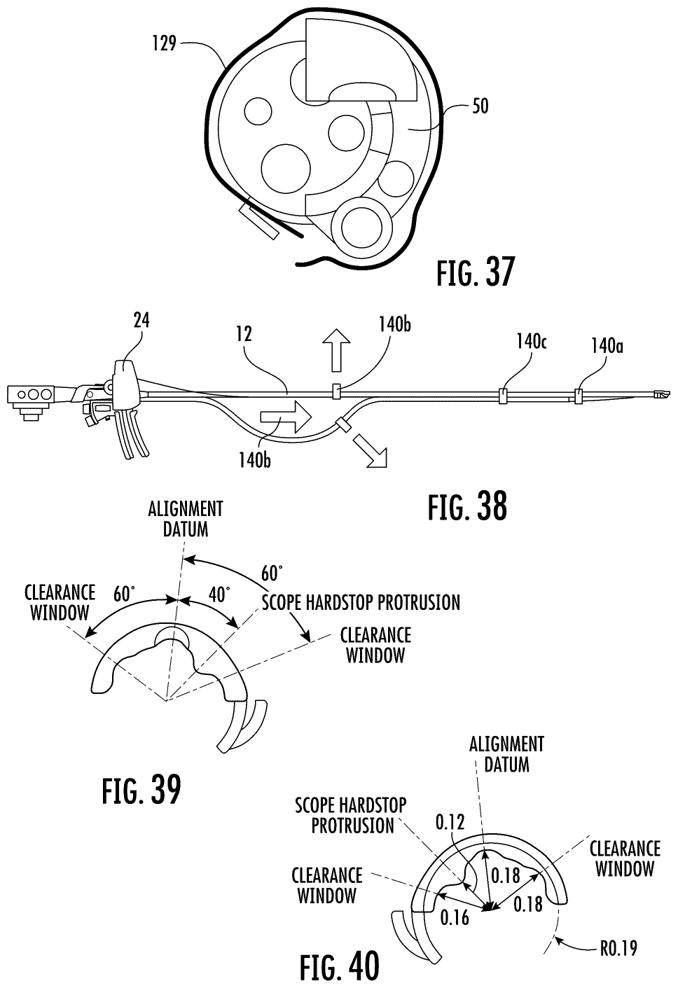

The suturing assembly is then released from over the endoscope by cutting, e.g., with a scissors 190, the two bands of tape 129 coupled to the hooks 120, 122 that secure the endoscope 12 relative to the cap assembly 50, and then unwrapping the tape 129 over the opening of the cap clip 102, as shown by both FIG. 36 and FIG. 37. Then, referring to FIG. 38, the tape strips 140b, 140c, 140a are broken apart, in turn, by applying force in the direction of arrow 174 to break each respective piece of tape at its perforation 152 (FIG. 19); as each tape strip was not directly adhered to the endoscope but only to itself and the sheath 89, the endoscope can be released once the strips are broken. The proximal handle 24 is then fully released from the endoscope 12.

In view of the above, in accordance with various principles of the present disclosure, an example of an embodiment of an endoscopic apparatus for use with an endoscope has a proximal handle assembly; a distal cap assembly; and a central portion adapted to extend along the endoscope between the handle assembly and the cap assembly. In some aspects, the handle assembly defines a recess in which the distal end of the endoscope is received. In some embodiments, the handle includes at least one hook at one side of the recess for receiving a tensile member that wraps around a portion of the cap assembly and the endoscope.

In some embodiments, the at least one hook is identified with a contrasting color relative to a majority of the cap assembly.

Additionally or alternatively, in some embodiments, the distal cap assembly includes a distal stop against which the distal end of the endoscope is intended to be abutted. In some embodiments, the distal stop includes indicia relative to which a feature of the endoscope is intended to be rotationally aligned. In some embodiments, the indicia include a contrasting color relative to a majority of the cap assembly. In some embodiments the indicia on the distal stop and the at least one hook are identified with a common color. In some embodiments, the feature of the endoscope with which the indicia are to be aligned is an instrument channel of the endoscope.

Additionally or alternatively, in some embodiments, the cap assembly is a suturing assembly including a movably mounted needle holder. In some aspects, operation of the proximal handle assembly moves the needle holder on the cap assembly.

In accordance with various principles of the present disclosure, an example of an embodiment of an endoscopic apparatus for use with an endoscope includes a proximal handle assembly; a distal cap assembly; and a central portion adapted to extend along the endoscope between the handle assembly and the cap assembly. In some aspects, the cap assembly defines a recess in which the distal end of the endoscope is received. Additionally or alternatively, the cap assembly includes two longitudinally spaced apart hooks at one side of the recess.

Additionally or alternatively, in some embodiments, each of the hooks are identified by a contrasting color relative to a color of a majority of the cap assembly.

Additionally or alternatively, in some embodiments, each of the hooks are identified by different hook indicia. In some embodiments, the hook indicia indicate an order of operation in which the hooks are intended to be used. In some embodiments, the hook indicia include numbers.

Additionally or alternatively, in some embodiments, the distal cap assembly includes a resilient clip that defines the recess.

Additionally or alternatively, in some embodiments the distal cap assembly includes a distal stop against which the distal end of the endoscope is intended to be abutted, the stop including contrasting alignment indicia relative to which a feature of the endoscope is intended to be rotationally aligned.

Additionally or alternatively, in some embodiments the distal cap assembly includes a distal stop against which the distal end of the endoscope is intended to be abutted, the stop including contrasting alignment indicia relative to which an instrument channel of the endoscope is intended to be rotationally aligned. In some embodiments the distal stop includes first and second recesses, and the alignment indicia is provided at the first recess.

In some embodiments, the cap assembly is a suturing assembly including a movably mounted needle holder, and operation of the proximal handle assembly moves the needle holder on the cap assembly.

In accordance with various principles of the present disclosure, an example of an embodiment of a securing system for use with an endoscope includes an endoscopic apparatus including a proximal handle assembly, a distal cap assembly; a central portion adapted to extend along the endoscope between the handle assembly and the cap assembly; and a spool of tape. In some embodiments, the distal cap assembly defines a recess in which the distal end of the endoscope is received and a hook at a side of the recess. In some embodiments, the spool of tape has a free end provided with a preformed loop adapted to be received over the hook. In some embodiments, the loop is adapted to be attached over the hook. In some embodiments, the tape is adapted to be wrapped about a portion of the cap assembly and the endoscope to couple the cap assembly and endoscope relative to each other.

In some embodiments, the system further includes a tape applicator including a handle and an extension with a spool mount. In some embodiments, the spool is coupled to the spool mount. In some embodiments, the spool is removably coupled to the spool mount.

In some embodiments, the cap assembly includes at least two longitudinally displaced hooks. In some embodiments, the securing system includes at least two spools of tape.

In some embodiments, the hook is identified by a contrasting color from a color of a majority of the cap assembly.

In some embodiments, the cap assembly includes at least two hooks. In some embodiments, each of the hooks is identified by different hook indicia. In some embodiments, the hook indicia identify an order of operation in which the hooks are intended to be used. In some embodiments, the hook indicia include a contrasting color from a majority of the cap assembly.

In some embodiments, the cap assembly is a suturing assembly including a movably mounted needle holder. In some embodiments, operation of the proximal handle assembly moves the needle holder on the cap assembly.

In accordance with various principles of the present disclosure, a method includes coupling a cap assembly of an endoscopic apparatus to an endoscope having a distal end. In some aspects, the cap assembly defines a recess in which the distal end of the endoscope is received. Additionally or alternatively, the cap assembly has first and second longitudinally displaced hooks at one side of the recess. In accordance with various principles of the present disclosure, the method includes inserting the distal end of the endoscope into the recess; providing a first length of tape having a first loop at a free end; coupling the first loop to the first hook; and wrapping at least a portion of the first length of tape about the cap assembly and the distal end of the endoscope.

In some aspects, the method further includes providing a second length of tape having a second loop at a free end; coupling the second loop to the second hook; and wrapping at least a portion of the second length of tape about the cap assembly and the distal end of the endoscope. In some aspects, the first hook is distally displaced from the second hook. In some aspects, the endoscopic apparatus includes a proximal handle and a central longitudinal sheath portion extending between the handle and the cap assembly. In some aspects, the central longitudinal portion includes a concave recess along its length. In some aspects, the method further includes inserting the endoscope into the recess of the central longitudinal sheath portion; and securing the central longitudinal sheath portion to the endoscope. In some aspects, the sheath portion is secured to the endoscope with a plurality of longitudinally spaced apart pieces of tape positioned at least partially circumferentially about the sheath portion and the endoscope. In some aspects, the central longitudinal sheath portion is crescent shaped and defines at least one lumen. In some aspects, at least one lumen includes a first lumen through which a flexible first catheter extends to the cap assembly, and a second lumen through which a flexible second catheter extends to the cap assembly. In some aspects, the cap assembly includes an actuation arm movably mounted thereon. In some aspects, the central longitudinal sheath portion includes a third lumen through which an actuation assembly extends. In some aspects, the actuation assembly is coupled between the proximal handle and the actuation arm and adapted for moving the actuation arm on the cap assembly upon operation of the proximal handle. In some aspects, the endoscopic apparatus includes a pull string having a proximal end and a distal end, the pull string extending from the proximal handle, through the central longitudinal sheath portion, and fixed to the cap assembly. In some aspects, the proximal end of the pull string is retracted relative to the proximal handle, and the distal end of the pull string is tensioned to cause the first and second catheters to flex and draw the cap assembly into retroflexion.

In some aspects, the cap assembly is a suturing assembly including a movably mounted needle holder.

In accordance with various principles of the present disclosure, an example of an embodiment of an endoscopic apparatus for use with an endoscope includes a proximal handle assembly; a distal cap assembly; a sheath portion; and a securing system. In some aspects, the cap assembly defines an assembly for positioning adjacent the distal end of the endoscope. In some aspects, the sheath portion defines a longitudinal recess. In some aspects, the sheath portion is adapted to extend along a portion of an exterior of the endoscope between the handle assembly and the cap assembly. In some aspects, the sheath portion is adapted to extend at least partially about the endoscope at the recess. In some aspects, the recess has a first side and a second side. In some aspects, the securing system is configured to secure the central portion to the exterior of the endoscope during in vivo operation of the endoscope. In some aspects, the securing system includes a plurality of longitudinally spaced apart lengths of tape pre-applied to the sheath portion on the first side of the recess. In some aspects, at least some of or each of the lengths of tape has an adhesive first portion at which the tape is adhered to the sheath on the first side of the recess, and a non-adhesive second portion extending from the first portion. In some aspects, at least some of or each of the lengths of tape has an adhesive third portion extending from the second portion and terminating in a free end.

In some aspects, the securing system further includes a removable non-adhesive protective strip over the third portion.

In some aspects, the protective strip extends into a tab with a free end protruding between the free end and the first portion.

Additionally or alternatively, the protective strip extends into an L-shaped non-adhesive tab. Additionally or alternatively, the tab has indicia indicating a direction in which the tab should be pulled to expose the third portion.

Additionally or alternatively, the protective strip has a contrasting color to the sheath portion.

In some aspects, each length of tape has a perforation between the, at, or near a junction of the second and third portions of the tape.

In some aspects, the securing system includes a first length of tape pre-applied at a distal end of the sheath portion, a second length of tape pre-applied at a central portion of the sheath portion, and a third length of tape pre-applied to the sheath portion between the first and second lengths of tape.

In some aspects, the sheath portion defines at least one lumen in a wall of the sheath portion.

In some aspects, the cap assembly is a suturing assembly including a movably mounted needle holder. In some aspects, operation of the proximal handle assembly moves the needle holder on the cap assembly.

In accordance with various principles of the present disclosure, a method of securing an endoscopic apparatus to an endoscope includes securing an endoscope having an external sheath to an endoscope. In some aspects, the endoscope has a circumference and a first length, and the sheath has a second length configured to extend about at least a portion of the circumference of the endoscope along at least a portion of the first length of the endoscope. In some aspects, the method includes providing the sheath with pre-applied portions of tape along second length; extending the sheath about the portion of the circumference of the endoscope along the first length; and wrapping the pre-applied portions of tape about a periphery of the endoscope and sheath to secure the sheath to the endoscope.

In some aspects, at least one or each pre-applied portion of tape includes an adhesive first portion at which the portion of tape is adhered to the sheath. Additionally or alternatively, at least one or each pre-applied portion of tape includes a non-adhesive second portion extending from the first portion. Additionally or alternatively, at least one or each pre-applied portion of tape includes an adhesive third portion extending from the second portion and terminating in a free end. Additionally or alternatively, at least one or each pre-applied portion of tape includes a removable protective strip over the third portion. In some aspects, the method further includes removing the removable protective strip from other the third portion, extending the second portion into contact with the endoscope, and adhering the third portion into contact with the sheath on an opposite side endoscope relative to the first portion.

In some aspects, the sheath defines a recess, the endoscope is inserted into the recess, and the first and third portions of the tape are located on opposite sides of the recess.

In some aspects, each pre-applied portion of tape includes a perforation. In some aspects, the method further includes releasing the endoscope from the sheath by applying a force to break the tape at the perforation. In some aspects, the tape is broken without cutting. In some aspects, the tape is broken by applying a force parallel to a longitudinal axis of the endoscope. In some aspects, the cap assembly is a suturing assembly including a movably mounted needle holder.

In accordance with various principles of the present disclosure, an example of an embodiment of an endoscopic apparatus for use with an endoscope includes a proximal handle assembly; a distal cap assembly; a sheath; a first catheter; a second catheter; and a transmission cable. In some aspects, the distal cap assembly is adapted to be positioned adjacent the distal end of the endoscope. In some aspects, the cap assembly has an end effector movable thereon. In some aspects, the sheath is a crescent-shaped flexible sheath. Additionally or alternatively, the sheath has a longitudinal side-opening recess sized to receive a portion of the endoscope between the handle assembly and the cap assembly. Additionally or alternatively, the sheath defines a first lumen, a second lumen, and a third lumen. In some aspects, the first catheter extends in and distally beyond the first lumen to the cap assembly. In some aspects, the second catheter extends in and distally beyond the second lumen to the cap assembly. In some aspects, the transmission cable extends from the handle assembly. In some aspects, the transmission assembly extends from the handle assembly into the third lumen. In some aspects, the transmission assembly extends distally beyond the third lumen to the cap assembly. In some aspects, the transmission assembly is operably coupled to the end effector. In some aspects, operation of the handle assembly moves the transmission cable to operate the end effector.

In some aspects, the cap assembly is a suturing assembly and the end effector is a needle holder.

In accordance with various principles of the present disclosure, an example of an embodiment of an endoscopic apparatus is configured for use with an endoscope having a proximal end and a distal end, an instrument channel, and a lens. In accordance with various principles of the present disclosure, the endoscopic apparatus includes a proximal handle; a distal cap assembly; and a central portion extending between the handle and cap assembly. In some aspects, the distal cap assembly defines an assembly for interacting with tissue. In some aspects, the distal cap assembly is adapted to be positioned adjacent the distal end of the endoscope. In some aspects, the distal cap assembly includes a clip to receive the distal end of the endoscope. In some aspects, the distal cap assembly includes a stop to limit distal travel of the endoscope. In some aspects, the stop has indicia against which a feature of the endoscope is rotationally aligned to ensure appropriate orientation of the lens and instrument channel of the endoscope without obstruction thereof. In some aspects, the central portion extends between the handle and cap assembly.

In some aspects, the stop is adapted to function with endoscopes from multiple manufacturers.

In some aspects, the cap assembly is a suturing assembly including a movably mounted needle holder. In some aspects, operation of the proximal handle moves the needle holder on the cap assembly.

In accordance with various principles of the present disclosure, an example of an embodiment of an endoscopic apparatus for use with an endoscope includes a proximal handle assembly; a distal cap assembly; a sheath; at least one catheter; and a pull string. In some aspects, the distal cap assembly is adapted to couple to the distal end of the endoscope spaced from the handle assembly by a first length. In some aspects, the sheath is a flexible sheath. In some aspects, the sheath has a proximal end and a distal end, the proximal end attached to the handle assembly. In some aspects, the sheath extends a second length less than the first length. In some aspects, the difference between the first length and the second length defines a gap. In some aspects, the sheath is adapted to extend at least partially about an exterior of the endoscope. In some aspects, the sheath defines at least one lumen. In some aspects, the at least one catheter extends through the at least one lumen from the proximal end of the sheath. In some aspects, the at least one catheter extends beyond the distal end of the sheath. In some aspects, the at least one catheter is fixed to the cap assembly. In some aspects, the pull string has a proximal end and a distal end. In some aspects, the pull string extends from the proximal end of the sheath, through the at least one lumen, out of the distal end of the sheath. In some aspects, the distal end of the pull string is fixed to the cap assembly. In some aspects, the proximal end of the pull string extends from an opening in the handle assembly. In some aspects, when the proximal end of the pull string is retracted relative to the handle, the distal end of the pull string is tensioned to cause the catheter to flex and draw the cap assembly into retroflexion across the gap.

In some aspects, the distal cap assembly is a suturing assembly including a needle movable relative to the at least one catheter. In some aspects, the pull string extends through a common lumen with a transmission cable for operating the suturing assembly.

In some aspects, the apparatus includes a pull handle attached to the proximal end of the pull string. In some aspects, the proximal handle assembly is adapted to releasably store the pull handle.

In some aspects, the apparatus further includes a locking system to temporarily retain the pull string under tension. In some aspects, the proximal handle assembly includes the locking system. In some aspects, the locking system includes a post having a seat and a washer provided over the post on the seat. In some aspects, the proximal end of the pull string is retained in position on the post. In some aspects, the proximal end of the pull string is retained in position on the post when wrapped around the post between the seat and the washer. In some aspects, the washer is made of a resilient material.

In accordance with various principles of the present disclosure, an example of an embodiment of an endoscopic system includes an endoscope and an external instrument channel device couplable to the endoscope. In some aspects, the endoscope has a proximal end and a distal end and an outer surface extending between the proximal and distal ends. In some aspects, the proximal end has a first handle. In some aspects, the distal end is adapted to retroflex upon actuation of the proximal end. In some aspects, the instrument channel device has a second handle. In some aspects, the instrument channel device has a sheath adapted to be coupled in close proximity to the outer surface of the endoscope between the proximal and distal ends. In some aspects, the instrument channel device has a cap removably coupled to the distal end of the endoscope. In some aspects, the instrument channel device has an instrument channel extending from the distal end of the sheath to the cap. In some aspects, the instrument channel device has a pull string extending from the second handle, through the sheath, and fixed relative to the cap. In some aspects, when the pull string is placed under tension, the cap is biased into a retroflex position about the instrument channel.

In some aspects, the cap is part of a suturing assembly.

In some aspects, the system further includes a pull handle attached to the proximal end of the pull string. In some aspects, the external instrument channel device includes a proximal handle assembly. In some aspects, the proximal handle assembly is adapted to releasably store the pull handle.

In some aspects, the system further includes a locking system to temporarily retain the pull string under tension. In some aspects, the external instrument channel device includes a proximal handle assembly, and the proximal handle assembly is provided with the locking system. In some aspects, the locking system includes a post having a seat and a washer provided over the post on the seat. In some aspects, the proximal end of the pull string is retained in position on the post. In some aspects, the proximal end of the pull string is retained in position on the post when wrapped around the post between the seat and the washer.

The suturing assemblies described above are adapted for use with an endoscope that does not necessarily have at least two instrument channels. As such, the suturing system can be used with smaller endoscopes that are available in many surgical settings and which can be more easily advanced through a natural orifice. In addition, as indicated aspects of the system can be used in other surgical treatment settings other than for suturing.

There have been described and illustrated herein embodiments of a suturing system as well as a surgical treatment system, as well as methods of using the same. While particular embodiments of the invention have been described, it is not intended that the invention be limited thereto, as it is intended that the invention be as broad in scope as the art will allow and that the specification be read likewise. Thus, while particular instruments and devices for advancement through the first and second lumen have been disclosed, it will be appreciated that other instruments can also be used through such lumen for like or even different purpose. Also, while the treatment system has been particularly described with respect to a cap assembly having an end effector in the form of a needle arm that carries a needle, it is recognized that alternatively one or more movable end effectors with other structure and purpose can be provided to the cap assembly. Also, while a tissue anchor in the form of a needle assembly has been described, the end effector can deploy different types of tissue anchors, including, e.g., clips. In addition, while a particular needle assembly has been described, other needle assemblies can similarly be used. Also, the size and instrument channel features of the endoscope with which the system is used is not critical, it is appreciated that various prior art systems cannot be properly used in a suturing operation in conjunction with endoscopes having fewer than two instrument channels, one for receiving a needle exchange device and the other for receiving a tissue retractor, whereas the present system is capable of complete operation without the provision of any channels through the endoscope. Further, while it is indicated that various features described herein are not limited to suturing applications, such as the retroflex system, it is specifically recognized that the retroflex system may be used in association with a cap assembly adapted to provide various other surgical applications including, but not limited to, staplers, clip appliers, band ligators, tissue manipulating instruments, cutting instruments, forceps, biopsy instruments, injection devices, as well as cap assemblies that have no significant function other than to support external catheters for passthrough of instruments. It will therefore be appreciated by those skilled in the art that yet other modifications could be made to the provided invention without deviating from its scope as claimed.

It is to be understood by one of ordinary skill in the art that the present discussion is a description of illustrative examples of embodiments only, and is not intended as limiting the broader aspects of the present disclosure.

All apparatuses and methods discussed herein are examples of apparatuses and/or methods implemented in accordance with one or more principles of this disclosure. These examples are not the only way to implement these principles but are merely examples, not intended as limiting the broader aspects of the present disclosure. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. It should be apparent to those of ordinary skill in the art that variations can be applied to the disclosed devices, systems, and/or methods, and/or to the sequence of steps of the method described herein without departing from the concept, spirit, and scope of the disclosure. It will be appreciated that various features described with respect to one embodiment typically may be applied to another embodiment, whether or not explicitly indicated. The various features hereinafter described may be used singly or in any combination thereof. Therefore, the present invention is not limited to only the embodiments specifically described herein, and all substitutes and modifications apparent to those skilled in the art are deemed to be within the spirit, scope, and concept of the disclosure as defined by the appended claims. Various further benefits of the various aspects, features, components, and structures of devices and systems such as described above, in addition to those discussed above, may be appreciated by those of ordinary skill in the art.

The foregoing discussion has broad application and has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. While the disclosure is presented in terms of embodiments, it should be appreciated that the various separate features of the present subject matter need not all be present in order to achieve at least some of the desired characteristics and/or benefits of the present subject matter or such individual features. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. As used in this specification and the appended claims, the term “or” is generally employed in its sense including “and/or” unless the content clearly dictates otherwise. As used herein, the conjunction “and” includes each of the structures, components, features, or the like, which are so conjoined, unless the context clearly indicates otherwise, and the conjunction “or” includes one or the others of the structures, components, features, or the like, which are so conjoined, singly and in any combination and number, unless the context clearly indicates otherwise. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, engaged, joined, etc.) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the terms “comprises”, “comprising”, “includes”, and “including” do not exclude the presence of other elements, components, features, groups, regions, integers, steps, operations, etc. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. An auxiliary medical system for use with another medical device having a proximal end and a distal end, the auxiliary system comprising:
a flexible elongate member having a device-engaging section sized, shaped, configured, and/or dimensioned to engage the other medical device;
a distal mount configured to engage the other medical device proximate to a distal end of the other medical device; and
a proximal mount configured to engage the other medical device proximate to a proximal end of the other medical device.

2. The auxiliary medical system of claim 1, wherein said flexible elongate member comprises a sheath having a length selected to extend at least partially along the length of the other medical device.

3. The auxiliary medical system of claim 2, wherein said sheath has a device-engaging section with a concave shape configured to receive a convexly curved outer surface of the other medical device.

4. The auxiliary medical system of claim 1, wherein said distal mount is configured to mount on a distal end of a medical scope.

5. The auxiliary medical system of claim 4, wherein said distal mount includes a distal stop against which the distal end of the medical scope is abutted when fitted within said distal mount, the distal stop including indicia relative to which a feature of the medical scope is rotationally alignable.

6. The auxiliary medical system of claim 1, wherein said flexible elongate member has one or more auxiliary working channels extending therethrough.

7. The auxiliary medical system of claim 1, further comprising one or more attachment strips having a first end attachable to said flexible elongate member and wrappable around said flexible elongate member and the other medical device to secure said auxiliary medical system with respect to the other medical device.

8. The auxiliary medical system of claim 7, further comprising a tape system including said one or more attachment strips.

9. The auxiliary medical system of claim 8, wherein said tape system further comprises a tape applicator.

10. The auxiliary medical system of claim 7, wherein said distal mount includes hooks and at least one of said one or more attachment strips includes holes for securing over said hooks.

11. The auxiliary medical system of claim 1, wherein said proximal mount is configured to be coupled with a handle of the other medical device.

12. An auxiliary medical system for use with another medical device having a proximal end and a distal end, the auxiliary system comprising:
an auxiliary device configured to be mounted with respect to the other medical device; and
at least one mount configured to engage the other medical device and an end of an attachment strip configured to be wrapped around said auxiliary device and the other medical device.

13. The auxiliary medical system of claim 12, wherein said at least one mount is an end cap configured to be mounted with respect to a distal end of the other medical device.

14. The auxiliary medical system of claim 12, wherein said at least one mount includes at least one hook configured to engage a hole in an end of an attachment strip configured to be wrapped around said auxiliary device and the other medical device.

15. The auxiliary medical system of claim 14, further comprising at least one attachment strip having a first end with a hole defined therethrough shaped to extend over said at least one hook of said at least one mount to secure the first end of said at least one attachment strip with respect to said at least one mount.

16. A method of engaging an auxiliary device with respect to another medical device, said method comprising:
engaging a section of the auxiliary device with the other medical device; and
securing the auxiliary device with respect to the other medical device with two or more attachment strips by attaching a free end of the attachment strips to the auxiliary device and wrapping the attachment strips around the auxiliary device and the other medical device.

17. The method of claim 16, wherein the auxiliary device is a sheath, and engaging a section of the sheath with the other medical device comprises engaging a concave section of the sheath with a convex surface of the other medical device.

18. The method of claim 16, wherein:
the other medical device is a medical scope;
the auxiliary device further includes a distal mount configured to be mounted on a distal end of the medical scope; and
said method further comprising orienting features on the distal mount of the auxiliary device with respect to features of the medical scope.

19.

20. The method of claim 16, wherein securing the auxiliary device with respect to the other medical device further comprises tightening at least one of the attachment strips while wrapping the at least one attachment strip after securing the free end of the at least one attachment strip with respect to the auxiliary device.

21. The method of claim 19, wherein the auxiliary device further includes a distal mount configured to be mounted with respect to a distal end of the other device, said method further comprising hooking a hole formed in the free end of the at least one of the attachment strips over a hook extending from the distal mount.
